(19) United States
(12) Patent Application Publication
(10) Pub. No.: US 2004/0259889 A1
(43) Pub. Date: Dec. 23, 2004

(54) Methods of treating pulmonary disease

(76) Inventors: Glenn J Smits, Oxford, MA (US); Francis G Spinale, Charleston, SC (US)

Correspondence Address: James F Haley, Fish & Neave, 1251 Avenue of the Americas, New York, NY, 10020-1104, US

(21) Appl. No.: 10/488,573

(22) PCT Filed: Sep. 6, 2002
(86) PCT No.: PCT/US02/28580

Related U.S. Application Data
(60) Provisional application No. 60/317,908, filed on Sep. 6, 2001.

(51) Int. Cl. A61K 31/522

(52) U.S. Cl. 514/263.33; 514/263.34

(57) ABSTRACT

Methods useful for reducing pulmonary vasoconstriction or improving pulmonary hemodynamics in a patient are disclosed. More particularly, this invention relates to administering A₁ adenosine receptor antagonists to reduce pulmonary vasoconstriction and improve pulmonary hemodynamics.

### TECHNICAL FIELD OF THE INVENTION

This invention relates to cardiology, medicinal chemistry and pharmacology. More particularly, it relates to A₁ adenosine receptor antagonists and reducing pulmonary vasoconstriction or improving pulmonary hemodynamics.

### BACKGROUND OF THE INVENTION

Pulmonary diseases can be life-threatening. Pulmonary edema and pulmonary hypertension are two such diseases. Pulmonary edema may be caused by a variety of physical conditions, e.g., altered alveolar-capillary membrane permeability, acute respiratory distress syndrome, increased pulmonary capillary pressure, decreased oncotic pressure, and lymphatic insufficiency. The causes for pulmonary hypertension include but are not limited to hypoxemia, respiratory system disorders, heart disease, thrombotic disease and embolic disease.

Conventional treatment of these pulmonary diseases involves drugs such as calcium channel blockers, diuretics, morphine sulfate, vasodilators such as nitrates, positive inotropic agents, prostacyclin and anticoagulants.

Adenosine is an intracellular and extracellular messenger generated by all cells in the body. It is also generated extracellularly by enzymatic conversion. Adenosine receptors are divided into four known subtypes (i.e., A₁, A₂ₐ, A_{2b} and A₃) based on their relative affinity for various adenosine receptor ligands and by sequence analysis of genes encoding these receptors. The activation of each of the subtypes elicits unique and sometimes opposing effects. Adenosine is associated with coronary and systemic vasodilation. The presence of adenosine receptors and the function of these receptors in pulmonary vasculature have been demonstrated in several species, including humans (see, e.g., Kucukhuseyin et al., J. Basic Clin. Physiol. Pharmacol., 8(4), pp. 287-299 (1997); Hong, J. L., et al., J. Physiol., 508(Pt1), pp. 109-118 (1998)). These studies indicate both A₁ and A₂ subtype receptors are present in the pulmonary vasculature. Activation of A₂ receptors leads to dilatation and relaxation of these vessels (See, e.g., McCormack et al., Am. J. Physiol., 256(1 Pt 2), pp. H41-H46 (1989); Szentmiklosi et al., Naunyn Schmiedebergs Arch. Pharmacol., 351(4), pp. 417-425 (1995); Cheng et al., Am. J. Physiol., 270(1 Pt 2), pp. H₂00-H₂07 (1996); Neely et al., Am. J. Physiol., 270(2 Pt 2), pp. H610-H619 (1996)). In contrast, these studies have shown that activation of A₁ receptors leads to constriction and contraction of these vessels, resulting in increased resistance to blood flow (see Neely et al., J. Pharmacol. Exp. Ther., 258(3), pp. 753-761 (1991); Broadly et al., J. Auton. Pharmacol., 16(6), pp. 363-366 (1996); see also, Szentmiklosi (1995), Cheng (1996) and Neely (1996), supra).

Despite the availability of a number of drugs to treat pulmonary diseases such as pulmonary edema and pulmonary hypertension, the median duration of survival after the diagnosis of primary pulmonary hypertension is 2.8 years (D'Alonzo et al., Ann. Intern. Med., 115, pp. 343-349 (1991)). Most of the current therapies involve non-specific vasodilation and reduction in peripheral (systemic) vascular resistance. These reductions in blood vessel tone in other parts of the body can result in reduced blood pressure that exacerbates the clinical situation by causing underperfusion of the tissues. Thus, there remains a need for new pharmaceutically acceptable compounds and compositions and improved methods for reducing vasoconstriction and improving pulmonary hemodynamics in patients suffering from pulmonary edema and pulmonary hypertension.

### SUMMARY OF THE INVENTION

Applicants have solved the above problem by discovering that A₁ adenosine receptor antagonists are capable of reducing pulmonary vasoconstriction and improving pulmonary hemodynamics without a concomitant reduction in peripheral vascular resistance. The invention relates to a method of reducing pulmonary vasoconstriction or improving pulmonary hemodynamics using A₁ adenosine receptor antagonists. The compounds useful in the methods of this invention exert their desirable effects through specifically antagonizing or blocking the A₁ adenosine receptor.

In some embodiments, the methods of this invention comprise administering to a patient a pharmaceutically effective amount of an A₁ adenosine receptor antagonist.

In some embodiments of the invention, the A₁ adenosine receptor antagonist employed is selected from the group consisting of:

a. a compound of formula I:

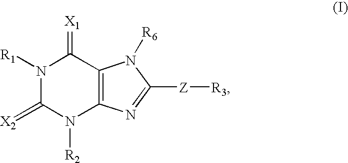

wherein R₁ and R₂ are independently selected from the group consisting of:

1) hydrogen;

2) alkyl, alkenyl of not less than 3 carbons, or alkynyl of not less than 3 carbons; wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and

3) aryl or substituted aryl;

R₃ is elected from the group consisting of:

1) a bicyclic, tricyclic or pentacyclic group selected from the group consisting of:

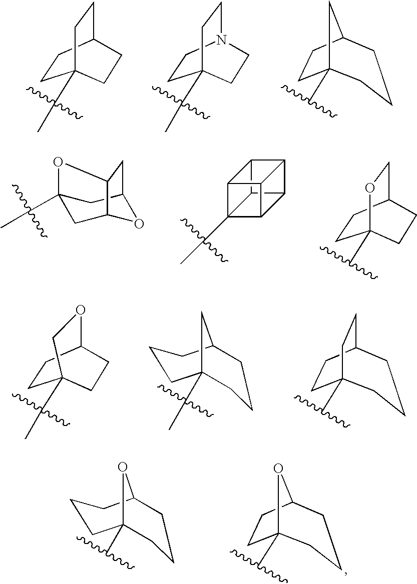

wherein the bicyclic or tricyclic group is either unsubstituted or functionalized with one or more substituents selected from the group consisting of:

a) alkyl, alkenyl, and alkynyl; wherein each alkyl, alkenyl, or alkynyl group is either unsubstituted or functionalized with one or more substituents selected from the group consisting of (amino)(R₅)acylhydrazinylcarbonyl, (amino) (R₅) acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and

b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, hydroxy, oximino, phosphate, substituted aralkylamino, substituted heterocyclyl, substituted heterocyclylsulfonylamino, sulfoxyacylamino, and thiocarbamoyl; and

2) the tricyclic group:

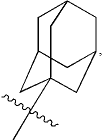

wherein the tricyclic group is functionalized with one or more substituents selected from the group consisting of:

a) alkyl, alkenyl, and alkynyl; wherein each alkyl, alkenyl, or alkynyl group is either unsubstituted or functionalized with one or more substituents selected from the group consisting of (amino) (R₅)acylhydrazinylcarbonyl, (amino) (R₅)acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and

b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, oximino, phosphate, substituted aralkylamino, substituted heterocyclyl, substituted heterocyclylsulfonylamino, sulfoxyacylamino, and thiocarbamoyl;

3) a bicyclic or tricyclic group selected from the group consisting of:

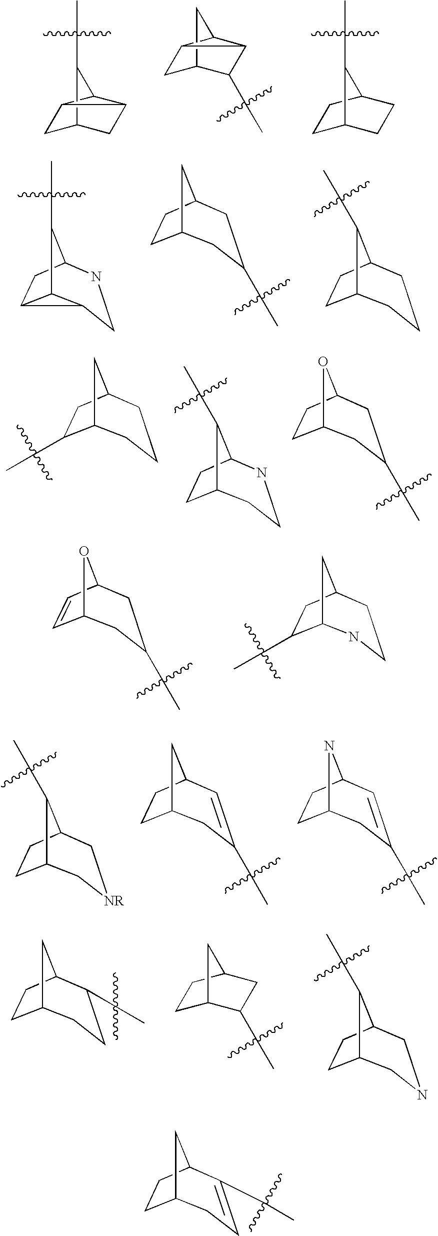

wherein the bicyclic or tricyclic group is either unsubstituted or fuctionalized with one or more substituents selected from the group consisting of:

a) alkyl, alkenyl, and alkynyl; wherein the alkyl, alkenyl, and alkynyl are either unsubstituted or functionalized with one or more substituents selected from the group consisting of alkoxy, alkoxycarbonyl, alkoxycarbonylaminoalkylamino, aralkoxycarbonyl, —R₅, dialkylamino, heterocyclylalkylamino, hydroxy, substituted arylsulfonylaminoalkylamino, and substituted heterocyclylaminoalkylamino;

b)acylaminoalkylamino, alkenylamino, alkoxycarbonyl, alkoxycarbonylalkylamino, alkoxycarbonylaminoacyloxy, alkoxycarbonylaminoalkylamino, alkylamino, amino, aminoacyloxy, carbonyl, —R₅, R₅-alkoxy, R₅-alkylamino, dialkylaminoalkylamino, heterocyclyl, heterocyclylalkylamino, hydroxy, phosphate, substituted arylsulfonylaminoalkylamino, substituted heterocyclyl, and sustituted heterocyclylaminoalkylamino;

R₄ is selected from the group consisting of hydrogen, C₁₋₄-alkyl, C₁₋₄-alkyl-CO₂H, and phenyl, wherein the C₁₋₄-alkyl, C₁₋₄-alkyl-CO₂H, and phenyl groups are either unsubstituted or functionalized with one to three substituents selected from the group consisting of halogen, —OH, —OMe, —NH₂, NO₂, benzyl, and benzyl functionalized with one to three substituents selected from the group consisting of halogen, —OH, —OMe, —NH₂, and —NO₂;

R₅ is selected from the group consisting of —CH₂COOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following:

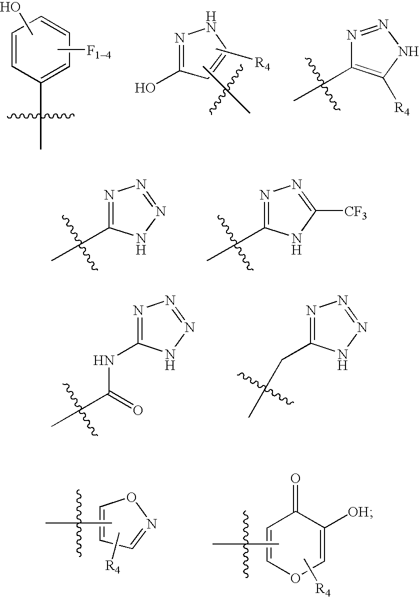

X₁ and X₂ are independently selected from the group consisting of O and S;

Z is selected from the group consisting of a single bond, —O—, —(CH₂)₁₋₃—, —O(CH₂)₁₋₂—, —CH₂OCH₂—, —(CH₂)₁₋₂O—, —CH═CHCH₂—, —CH═CH—, and —CH₂CH═CH—; and

R₆ is selected from the group consisting of hydrogen, alkyl, acyl, alkylsulfonyl, aralkyl, substituted aralkyl, substituted alkyl, and heterocycle; and

b. a compound of formula II or III:

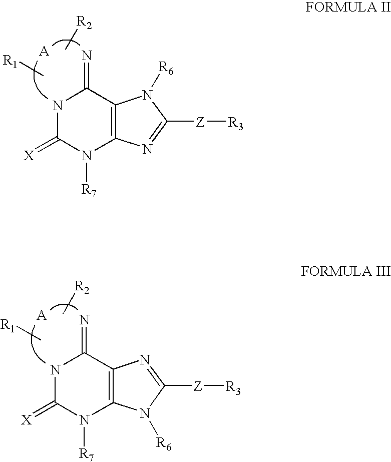

wherein R₁ and R₂ are independently selected from the group consisting of:

1) hydrogen;

2) alkyl, alkenyl or alkynyl, wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and

3) aryl or substituted aryl;

R₃ is selected from the group consisting of:

1) a bicyclic, tricyclic or pentacyclic group selected from the group consisting of:

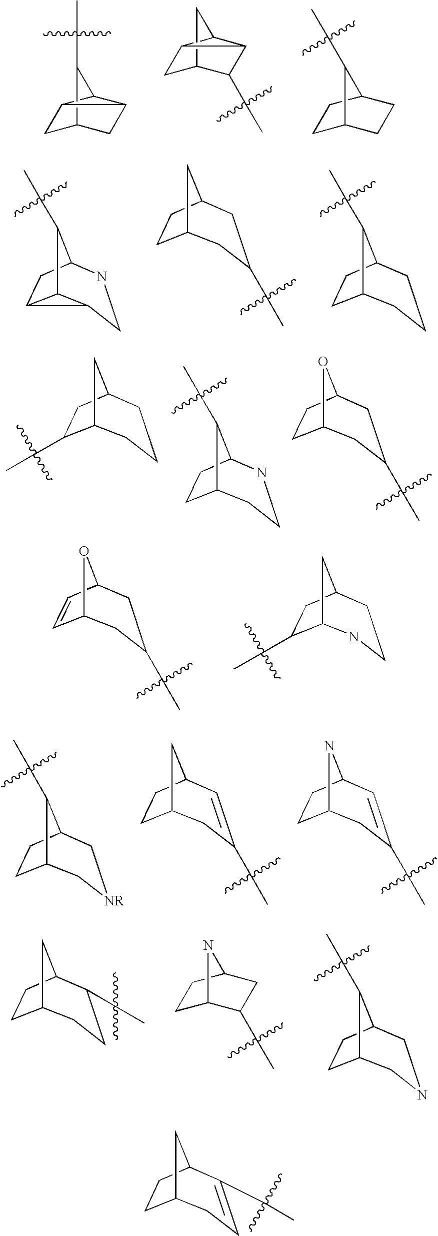

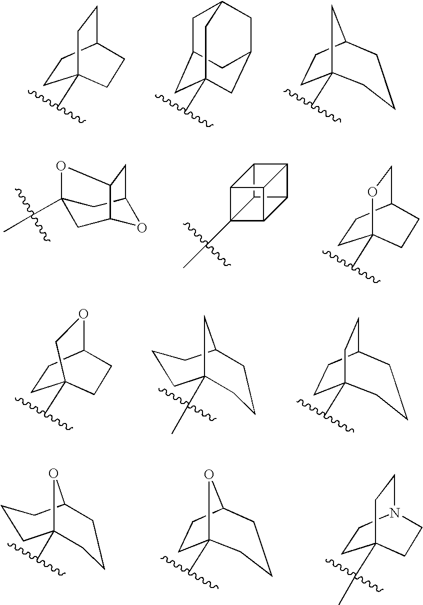

wherein the bicyclic, tricyclic or pentacyclic group is either unsubstituted or functionalized with one or more substituents selected from the group consisting of:

i) alkyl, alkenyl and alkynyl; wherein each alkyl, alkenyl or alkynyl group is either unsubstituted or functionalized with one or -more substituents selected from the group consisting of (alkoxycarbonyl)aralkylcarbamoyl, (amino)(R₅)acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl; and

ii) (alkoxycarbonyl)aralkylcarbamoyl, (amino)(R₅)acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl;

R₄ is selected from the group consisting of hydrogen, C₁₋₄-alkyl, C₁₋₄-alkyl-CO₂H, and phenyl, wherein the C₁₋₄-alkyl, C₁₋₄-alkyl-CO₂H, and phenyl groups are either unsubstituted or functionalized with one to three substituents selected from the group consisting of halogen, —OH, —OMe, —NH₂, NO₂, benzyl, and benzyl functionalized with one to three substituents selected from the group consisting of halogen, —OH, —OMe, —NH₂, and —NO₂;

R₅ is selected from the group consisting of —(CR₁R₂)ₙCOOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following:

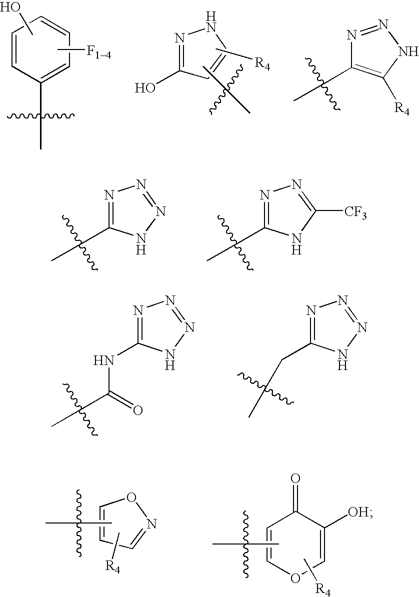

n=0, 1, 2 or 3;

A is selected from the group consisting of —CH═CH, —(CH)ₘ (CH)_{m,}, CH═CH—CH₂, and —CH₂—CH═CH;

m=1 or 2;

X is O or S;

Z is selected from the group consisting of a single bond, —O—, —(CH₂) n—, —O(CH₂)₁₋₂—, —CH₂OCH₂—, —(CH₂)₁₋₂O—, —CH═CHCH₂—, —CH═CH—, and —CH₂CH═CH—; and

R₆ is selected from the group consisting of hydrogen, alkyl, acyl, alkylsufonyl, aralkyl, substituted aralkyl, substituted alkyl, and heterocyclyl; and

R₇ is selected from the group consisting of:

1) hydrogen;

2) alkyl, alkenyl of not less than 3 carbons, or alkynyl of not less than 3 carbons; wherein said alkyl, alkenyl or alkynyl is either unsubstituted or functionalized with one or more substitutents selected from the group consisting of hydroxy, alkoxy, amino, alkylamino, dialkylamino, heterocyclyl, acylamino, alkylsulfonylamino, and heterocyclylcarbonylamino; and

3) aryl or substituted aryl;

4) alkylaryl or alkyl substituted aryl;

c. 8-(3-Oxa-tricyclo[3.2.1.0 ^{2,4}]oct-6-yl)-1,3-dipropyl-3,7-dihydro-purine-2,6-dione;

8-Bicyclo[2.2.1]hept-5-en-2-yl-1,3-dipropyl-3,7-dihydro-purine-2,6-dione;

7,8-dihydro-8-ethyl-2-(3-noradamantyl)-4-propyl-1H-imidazo[2,1-I]purine-5-(4H)-one;

8-(7-Hydroxy-3-noradamantyl)-1,3-dipropyl-3,7-dihydro-purine-2,6-dione;

8-(3-noradamantyl)-1,3-dipropyl-3,7-dihydro-purine-2,6-dione;

5-[8-(Isopropyl-methyl-amino)-9-methyl-9H-purin-6-ylamino]-bicyclo[2.2.1]heptan-2-ol;

1-[2-(2-Hydroxy-ethyl)-piperidin-1-yl]-3-(2-phenyl-pyrazolo[1,5-a]pyridin-3-yl)-propenone;

4-[6-Oxo-3-(2-phenyl-pyrazolo[1,5-a]pyridin-3-yl)-6H-pyridazin-1-yl]-butyric acid;

6-(2-Phenyl-pyrazolo[1,5-a]pyridin-3-yl)-2-[2-(1H-tetrazol-5-yl)-ethyl]-2H-pyridazin-3-one;

8-Cyclopentyl-1,3-dipropyl-3,7-dihydro-purine-2,6-dione (DPCPX);

8-(3-Oxo-cyclopentyl)-1,3-dipropyl-3,7-dihydro-purine-2,6-dione (Apaxifylline);

8-(1-Amino-cyclopentyl)-1,3-dipropyl-3,7-dihydro-purine-2,6-dione; and

8-Dicyclopropylmethyl-1,3-dipropyl-3,7-dihydro-purine-2,6-dione.

In some embodiments of this invention, the compound of formula I is selected from:

3-[4-(2,6-Dioxo-1,3-dipropyl-2,3,6,7-tetrahydro-1H-purin-8-yl)-bicyclo[2.2.2]oct-1-yl]-propionic acid;

3-[4-(2,6-Dioxo-1,3-dipropyl-2,3,6,7-tetrahydro-1H-purin-8-yl)-bicyclo[2.2.2]oct-1-yloxy]-propionic acid;

8-(1-Hydroxy-tricyclo[2.2.2.1.0^{2,6}]hept-3-yl)-1,3-dipropyl-3,7-dihydro-purine-2,6-dione; and

8-(4-Hydroxy-bicyclo[2.2.2]oct-1-yl)-1,3-dipropyl-3,7-dihydro-purine-2,6-dione.

In some embodiments of this invention, the compound of formula II or III is selected from:

7,8-Dihydro-8-isopropyl-2-(4-Hydroxy-bicyclo[2.2.2]oct-1-yl)-4-propyl-1H-imidazo[2,1-i]purin-5-(4H)-one; and

7,8-Dihydro-8-ethyl-2-(4-Hydroxy-bicyclo[2.2.2]oct-1-yl)-4-propyl-1H-imidazo[2,1-i]purin-5-(4H)-one.

In preferred embodiments, the A₁ adenosine receptor antagonist used in the method of this invention is selected from the group consisting of:

3-[4-(2,6-Dioxo-1,3-dipropyl-2,3,6,7-tetrahydro-1H-purin-8-yl)-bicyclo[2.2.2]oct-1-yl]-propionic acid;

8-(3-Oxa-tricyclo[3.2.1.0^{2,4}]oct-6-yl)-1,3-dipropyl-3,7-dihydro-purine-2,6-dione;

8-Bicyclo[2.2.1]hept-5-en-2-yl-1,3-dipropyl-3,7-dihydro-purine-2,6-dione;

7,8-Dihydro-8-isopropyl-2-(4-Hydroxy-bicyclo[2.2.2]oct-1-yl)-4-propyl-1H-imidazo[2,1-i]purin-5-(4H)-one;

7,8-Dihydro-8-ethyl-2-(4-Hydroxy-bicyclo[2.2.2]oct-1-yl)-4-propyl-1H-imidazo[2,1-i]purin-5-(4H)-one;

3-[4-(2,6-Dioxo-1,3-dipropyl-2,3,6,7-tetrahydro-1H-purin-8-yl)-bicyclo[2.2.2]oct-1-yloxy]-propionic acid;

8-(1-Hydroxy-tricyclo[2.2.1.0^{2,6}]hept-3-yl)-1,3-dipropyl-3,7-dihydro-purine-2,6-dione;

8-(4-Hydroxy-bicyclo[2.2.2]oct-1-yl)-1,3-dipropyl-3,7-dihydro-purine-2,6-dione; 7,8-dihydro-8-ethyl-2-(3-noradamantyl)-4-propyl-1H-imidazo[2,1-I]purine-5-(4H)-one; 8-(7-Hydroxy-3-noradamantyl)-1,3-dipropyl-3,7-dihydro-purine-2,6-dione;

8-(3-noradamantyl)-1,3-dipropyl-3,7-dihydro-purine-2,6-dione;

5-[8-(Isopropyl-methyl-amino)-9-methyl-9H-purin-6-ylamino]-bicyclo[2.2.1]heptan-2-ol;

1-[2-(2-Hydroxy-ethyl)-piperidin-1-yl]-3-(2-phenyl-pyrazolo[1,5-a]pyridin-3-yl)-propenone;

4-[6-Oxo-3-(2-phenyl-pyrazolo[1,5-a]pyridin-3-yl)-6H-pyridazin-1-yl]-butyric acid;

6-(2-Phenyl-pyrazolo[1,5-a]pyridin-3-yl)-2-[2-(1H-tetrazol-5-yl)-ethyl]-2H-pyridazin-3-one;

8-Cyclopentyl-1,3-dipropyl-3,7-dihydro-purine-2,6-dione (DPCPX);

8-(3-Oxo-cyclopentyl)-1,3-dipropyl-3,7-dihydro-purine-2,6-dione (Apaxifylline);

8-(1-Amino-cyclopentyl)-1,3-dipropyl-3,7-dihydro-purine-2,6-dione; and

8-Dicyclopropylmethyl-1,3-dipropyl-3,7-dihydro-purine-2,6-dione.

In more preferred embodiments, the A₁ adenosine receptor antagonist used in the method of this invention is selected from the group consisting of:

3-[4-(2,6-Dioxo-1,3-dipropyl-2,3,6,7-tetrahydro-1H-purin-8-yl)-bicyclo[2.2.2]oct-1-yl]-propionic acid;

8-(3-Oxa-tricyclo[3.2.1.0^{2,4}] oct-6-yl)-1,3-dipropyl-3,7-dihydro-purine-2,6-dione;

8-Bicyclo[2.2.1]hept-5-en-2-yl-1,3-dipropyl-3,7-dihydro-purine-2,6-dione;

7,8-Dihydro-8-isopropyl-2-(4-Hydroxy-bicyclo[2.2.2]oct-1-yl)-4-propyl-1H-imidazo[2,1-i]purin-5-(4H)-one;

7,8-Dihydro-8-ethyl-2-(4-Hydroxy-bicyclo[2.2.2]oct-1-yl)-4-propyl-1H-imidazo[2,1-i]purin-S-(4H)-one;

3-[4-(2,6-Dioxo-1,3-dipropyl-2,3,6,7-tetrahydro-1H-purin-8-yl)-bicyclo[2.2.2]oct-1-yloxy]-propionic acid;

8-(1-Hydroxy-tricyclo[2.2.1.0^{2,6}]hept-3-yl)-1,3-dipropyl-3,7-dihydro-purine-2,6-dione; and

8-(4-Hydroxy-bicyclo[2.2.2]oct-1-yl)-1,3-dipropyl-3,7-dihydro-purine-2,6-dione.

In other more preferred embodiments, the A₁ adenosine receptor antagonist used in the method of this invention is selected from:

3-[4-(2,6-Dioxo-1,3-dipropyl-2,3,6,7-tetrahydro-1H-purin-8-yl)-bicyclo[2.2.2]oct-1-yl)-propionic acid;

7,8-Dihydro-8-isopropyl-2-(4-Hydroxy-bicyclo[2.2.2]oct-1-yl)-4-propyl-1H-imidazo[2,1-i]purin-5-(4H)-one;

7,8-Dihydro-8-ethyl-2-(4-Hydroxy-bicyclo[2.2.2]oct-1-yl)-4-propyl-1H-imidazo[2,1-i]purin-5-(4H)-one;

3-[4-(2,6-Dioxo-1,3-dipropyl-2,3,6,7-tetrahydro-1H-purin-8-yl)-bicyclo[2.2.2]oct-1-yloxy]-propionic acid;

8-(1-Hydroxy-tricyclo[2.2.1.0^{2,6}]hept-3-yl)-1,3-dipropyl-3,7-dihydro-purine-2,6-dione; and

8-(4-Hydroxy-bicyclo[2.2.2]oct-1-yl)-1,3-dipropyl-3,7-dihydro-purine-2,6-dione.

In yet other more preferred embodiments, the A₁ adenosine receptor antagonist used in the method of this invention is selected from:

3-[4-(2,6-Dioxo-1,3-dipropyl-2,3,6,7-tetrahydro-1H-purin-8-yl)-bicyclo[2.2.2]oct-1-yl]-propionic acid;

7,8-Dihydro-8-isopropyl-2-(4-Hydroxy-bicyclo[2.2.2]oct-1-yl)-4-propyl-1H-imidazo[2,1-i]purin-5-(4H)-one;

7,8-Dihydro-8-ethyl-2-(4-Hydroxy-bicyclo[2.2.2]oct-1-yl)-4-propyl-1H-imidazo[2,1-i]purin-5-(4H)-one;

3-[4-(2,6-Dioxo-1,3-dipropyl-2,3,6,7-tetrahydro-1H-purin-8-yl)-bicyclo[2.2.2]oct-1-yloxy]-propionic acid.

In most preferred embodiments, the A₁ adenosine receptor antagonist used in the method of this invention is 3-[4-(2,6-Dioxo-1,3-dipropyl-2,3,6,7-tetrahydro-1H-purin-8-yl)-bicyclo[2.2.2]oct-1-yl]-propionic acid.

In some embodiments, the A₁ adenosine receptor antagonist used in the method of this invention is an antibody. Preferably, the antibody is directed to the ligand binding domain of the A₁ adenosine receptor.

In some embodiments, the A₁ adenosine receptor is administered to a human.

In some embodiments, the A₁ adenosine receptor antagonist used in the method of this invention is formulated together with a pharmaceutically suitable carrier into a pharmaceutically acceptable composition.

The invention is useful in the treatment of patients displaying signs or symptoms of pulmonary diseases. Examples of pulmonary diseases that can be treated by methods of the invention include pulmonary edema, pulmonary hypertension and a combination thereof.

In some embodiments of the invention, the method is used in the treatment of pulmonary edema accompanied by a condition selected from the group consisting of an imbalance of Starling forces, altered alveolar-capillary membrane permeability, lymphatic insufficiency.

In some embodiments of the invention, the method is used in the treatment of pulmonary hypertension accompanied by a condition selected from the group consisting of pulmonary arterial hypertension, pulmonary hypertension associated with disorders of the respiratory system or hypoxemia, pulmonary venous hypertension, pulmonary hypertension resulting from chronic thrombotic or embolic disease, pulmonary hypertension resulting from disorders directly affecting the pulmonary vasculature.

In some embodiments of the invention, the method is used in the treatment of a patient displaying signs or symptoms of pulmonary disease characterized by at least one of the following conditions: global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.

This invention also relates to a method of treating a patient displaying signs or symptoms of a pulmonary disease comprising the step of administering to the patient a pharmaceutically effective amount of a pharmaceutical composition comprising an A1 adenosine antagonist and a pharmaceutically acceptable carrier.

In some embodiments, the invention provides a method of treating a patient displaying signs or symptoms of a pulmonary disease selected from the group consisting of pulmonary edema, pulmonary hypertension and a combination thereof.

In some embodiments, the invention provides a method of treating a patient displaying signs or symptoms of pulmonary edema, wherein the pulmonary edema is accompanied by a condition selected from the group consisting of an imbalance of Starling forces, altered alveolar-capillary membrane permeability, lymphatic insufficiency.

In some embodiments, the invention provides a method of treating a patient displaying signs or symptoms of pulmonary hypertension, wherein the pulmonary hypertension is accompanied by a condition selected from the group consisting of pulmonary arterial hypertension, pulmonary hypertension associated with disorders of the respiratory system or hypoxemia, pulmonary venous hypertension, pulmonary hypertension resulting from chronic thrombotic or embolic disease, pulmonary hypertension resulting from disorders directly affecting the pulmonary vasculature.

In some embodiments, the invention provides a method of treating a patient displaying signs or symptoms of a pulmonary disease, wherein the pulmonary disease is characterized by at least one of the following conditions: global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.

### BRIEF DESCRIPTION OF THE DRAWINGS

FIG. 1 depicts the effect of an A₁ adenosine receptor antagonist BG9719, 1 mg/kg) on mean arterial pressure (MAP) and heart rate (HR). No change in heart rate or mean arterial pressure was noted following treatment with BG9719.

FIG. 2 depicts the effect of an A₁ adenosine receptor antagonist (BG9719, 1 mg/kg) on cardiac output (CO), pulmonary artery pressure (PAP), and pulmonary capillary wedge pressure (PCWP). No change in cardiac output was noted following treatment with BG9719. Pulmonary artery pressure decreased 30 minutes after treatment with BG9719 and remained depressed. Pulmonary capillary wedge pressure decreased 90 minutes after treatment with BG9719.

FIG. 3 depicts the measurement of Pulmonary Vascular Resistance (PVR) in pacing heart failure preparations after intravenous infusion of an A₁ adenosine receptor antagonist (BG9719, 1 mg/kg). PVR decreases by 38% from baseline and returns to baseline levels.

(+p<0.05 vs. baseline).

FIG. 4. Systemic vascular resistance (SVR) and Pulmonary Vascular Resistance were measured in pacing HF preparations at baseline and after intravenous infusion of an A₁ adenosine receptor antagonist (3-[4-(2,6-Dioxo-1,3-dipropyl-2,3,6,7-tetrahydro-1H-purin-8-yl)-bicyclo[2.2.2]oct-1-yl]-propionic acid (BG9928), 1 mg/kg). Results are expressed as % Change from Baseline. At 10 minutes post treatment with BG9928, PVR fell 18% from baseline levels while there was no change in SVR (+p<0.05 vs. baseline).

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. All publications, patents and other references mentioned herein are incorporated by reference.

Although methods and materials similar or equivalent to those described herein can be used in the practice or testing of the present invention, suitable methods and materials are described below. The materials, methods and examples are illustrative only, and are not intended to be limiting. Other features and advantages of the invention will be apparent from the detailed description and from the claims.

In order to further define this invention, the following terms and definitions are herein provided.

As used herein, “alkyl” group means a saturated aliphatic hydrocarbon group. An alkyl group can be straight or branched, and can have, for example, from 1 to 6 carbon atoms in a chain. Examples of straight chain alkyl groups include, but are not limited to, ethyl and butyl. Examples of branched alkyl groups include, but are not limited to, isopropyl and t-butyl.

As used herein, “alkenyl” group means an aliphatic carbon group that has at least one double bond. An alkenyl group can be straight or branched, and can have, for example, from 3 to 6 carbon atoms in a chain and 1 or 2 double bonds. Examples of alkenyl groups include, but are not limited to, allyl and isoprenyl.

As used herein, “alkynyl” group means an aliphatic carbon group that has at least one triple bond. An alkynyl group can be straight or branched, and can have, for example, from 3 to 6 carbon atoms in a chain and 1 to 2 triple bonds. Examples of alkynyl groups include, but are not limited to, propargyl and butynyl.

As used herein, “aryl” group means a phenyl or naphthyl group, or a derivative thereof. A “substituted aryl” group is an aryl group that is substituted with one or more substituents such as alkyl, alkoxy, amino, nitro, carboxy, carboalkoxy, cyano, alkylamino, dialkylamino, halo, hydroxy, hydroxyalkyl, mercaptyl, alkylmercaptyl, trihaloalkyl, carboxyalkyl, sulfoxy, or carbamoyl.

As used herein, “aralkyl” group means an alkyl group that is substituted with an aryl group. An example of an aralkyl group is benzyl.

As used herein, “cycloalkyl” group means an aliphatic ring of, for example, 3 to 8 carbon atoms. Examples of cycloalkyl groups include cyclopropyl and cyclohexyl.

As used herein, “acyl” group means a straight or branched alkyl-C(═O)— group or a formyl group. Examples of acyl groups include alkanoyl groups (e.g., having from 1 to 6 carbon atoms in the alkyl group). Acetyl and pivaloyl are examples of acyl groups. Acyl groups may be substituted or unsubstituted.

As used herein, “carbamoyl” group means a group having the structure H₂N—CO₂—. “Alkylcarbamoyl” and “dialkylcarbamoyl” refer to carbamoyl groups in which the nitrogen has one or two alkyl groups attached in place of the hydrogens, respectively. By analogy, “arylcarbamoyl” and “arylalkylcarbamoyl” groups include an aryl group in place of one of the hydrogens and, in the latter case, an alkyl group in place of the second hydrogen.

As used herein, “carboxyl” group means a —COOH group.

As used herein, “alkoxy” group means an alkyl-O— group in which “alkyl” is as previously described.

As used herein, “alkoxyalkyl” group means to an alkyl group as previously described, with a hydrogen replaced by an alkoxy group, as previously described.

As used herein, “halogen” or “halo” group means fluorine, chlorine, bromine or iodine.

As used herein, “heterocyclyl” group means a 5 to 10-membered ring structure, in which one or more of the atoms in the ring is an element other than carbon, e.g., N, O, S. A heterocyclyl group can be aromatic or non-aromatic, i.e., can be saturated, or can be partially or fully unsaturated. Examples of heterocyclyl groups include pyridyl, imidazolyl, furanyl, thienyl, thiazolyl, tetrahydrofuranyl, tetrahydropyranyl, morpholinyl, thiomorpholinyl, indolyl, indolinyl, isoindolinyl, piperidinyl, pyrimidinyl, piperazinyl, isoxazolyl, isoxazolidinyl, tetrazolyl, and benzimidazolyl.

As used herein, “substituted heterocyclyl” group means a heterocyclyl group wherein one or more hydrogens are replaced by substituents such as alkoxy, alkylamino, dialkylamino, carbalkoxy, carbamoyl, cyano, halo, trihalomethyl, hydroxy, carbonyl, thiocarbonyl, hydroxyalkyl or nitro.

As used herein, “hydroxyalkyl” means an alkyl group substituted by a hydroxy group.

As used herein, “sulfamoyl” group means the structure —S(O)₂NH₂. “Alkylsulfamoyl” and “dialkylsulfamoyl” refer to sulfamoyl groups in which the nitrogen has one or two alkyl groups attached in place of the hydrogens, respectively. By analogy, “arylsulfamoyl” and “arylalkylsulfamoyl” groups include an aryl group in place of one of the hydrogens and, in the latter case, an alkyl group in place of the second hydrogen.

As used herein, “antagonist” means a molecule that binds to a receptor without activating the receptor or triggering signal transduction. An antagonist competes with the endogenous ligand for the binding site, thereby interfering with stimulation or triggering of the receptor by the endogenous ligand. Antagonists include antibodies raised against the A₁ adenosine receptor and that block the adenosine binding site or prevent adenosine from binding to the receptor.

As used herein, “selective antagonist” means an antagonist that binds to a specific subtype of adenosine receptor with higher affinity than to other subtypes. For example a selective A₁ receptor antagonist has high affinity for A₁ receptors and has a) nanomolar binding affinity for the A₁ receptor subtype and b) at least 10 times, more preferably 50 times, and most preferably 100 times greater affinity for the A₁ receptor subtype that for another subtype.

As used herein, “antibody” means a polypeptide encoded by an immunoglobulin gene, genes, or fragments thereof. The immunoglobulin genes include the kappa, lambda, alpha, gamma, delta, epsilon and mu constant regions, as well as a vast number of immunoglobulin variable regions. Light chains are classified as either kappa or lambda. Heavy chains are classified as gamma, mu, alpha, delta, or epsilon, which in turn define the immunoglobulin classes IgG, IgM, IgA, IgD and IgE, respectively.

Antibodies exist for example, as intact immunoglobulins (consisting of two heavy chains and two light chains) or as a number of well-characterized fragments thereof. Such fragments include, but are not limited to, those produced by digestion with various proteases, those produced by chemical cleavage and/or chemical dissociation, and those produced recombinantly, so long as the fragment remains capable of specific binding to an antigen. Among these fragments are Fab, Fab′, F(ab′)₂, and single chain Fv (scFv) fragments. See Fundamental Immunology, Third Edition, W. E. Paul, ed. Raven Press, N.Y. (1993) for a detailed description of epitopes, antibodies and antibody fragments. Such Fab′ fragments may be obtained readily using conventional chemical synthesis or recombinant DNA technology. Thus, as used herein, the term antibody includes antibody fragments produced by the modification of whole antibodies or those synthesized de novo. Antibodies useful in the present invention are optionally derived from libraries of recombinant antibodies in phage or similar vectors (see, e.g., Huse et al., Science, 246, pp. 1275-81 (1989); Ward et al., Nature, 341, pp. 544-46 (1989); Vaughan et al., Nature Biotech., 14, pp. 309-14 (1996) which are incorporated herein by reference).

As used herein, “pharmaceutically effective amount” means the amount required to reduce or lessen the severity of vasoconstriction and/or improve pulmonary hemodynamics for some period of time. A pharmaceutically effective amount also means the amount required to improve the clinical symptoms of a patient.

As used herein, “pharmaceutically acceptable carrier or adjuvant” means to a non-toxic carrier or adjuvant that may be administered to a patient, together is with a compound of this invention, and which does not destroy the pharmacological activity thereof.

As used herein, “pulmonary edema” means a condition wherein the fluid is accumulated in the lungs. The clinical signs and symptoms of pulmonary edema can start as a primary manifestation of certain pathology or as an evolution of a pre-existing disease. Patients present themselves with a variety of symptoms including dyspnea, tachypnea, orthopnea, tachycardia, hypertension, thoracic oppression, cold extremities with or without cyanosis, cough with a frothy or pink sputum, extensive use of accessory muscles of respiration, moist rales with or without wheezing. Diagnosis of pulmonary edema is within ordinary skill in the art.

As used herein, “pulmonary hemodynamics” means the forces or mechanisms involved in circulating blood through the lungs. “Improved pulmonary hemodynamics” or “improving pulmonary hemodynamics” includes but is not limited to a reduction in pulmonary vascular resistance, reduction in pulmonary artery pressure, reduction in pulmonary capillary wedge pressure, increase in arterial oxygen saturation, reduction in ‘rales’, improvement in ‘shortness of breath’, and increase in exercise capacity when limited by pulmonary function.

As used herein, “pulmonary hypertension” means abnormally elevated blood pressure within the pulmonary circuit (pulmonary artery). Pulmonary hypertension may be secondary to another disease process or occur as a primary disease process known as primary pulmonary hypertension. Diagnosis of pulmonary hypertension is within ordinary skill in the art.

As used herein, “pulmonary vasoconstriction” means the narrowing of the lumen of blood vessels in the lungs, especially as a result of vasomotor action. Pulmonary vasoconstriction results in a decrease in the blood flow through the lungs or an increase in the resistance to blood flow through the pulmonary vasculature. “Reducing pulmonary vasoconstriction” includes a decrease in vasoconstriction or an increase in pulmonary vasodilation. “Pulmonary vasodilation” refers to a widening of the lumen of blood vessels. It is an increase in the internal diameter of a blood vessel that results from relaxation of smooth muscle within the wall of the vessel. This causes an increase in blood flow, and/or a decrease in pressure in the pulmonary artery pressure.

The present invention relates to methods for reducing pulmonary vasoconstriction or improving pulmonary hemodynamics in a patient. The methods include administering to a patient a pharmaceutically effective amount of an A₁ adenosine receptor antagonist.

Synthesis of the Adenosine Antagonist Compounds

Compounds useful in the invention may be prepared by conventional methods known in the art. For example, the synthesis of the compounds of formula I is described in International Publication Nos. WO 01/34604 and WO 01/34610.

The synthesis of the compounds 8-(3-Oxa-tricyclo[3.2.1.0^{2,4}] oct-6-yl)-1,3-dipropyl-3,7-dihydro-purine-2,6-dione and 8-Bicyclo[2.2.1]hept-5-en-2-yl-1,3-dipropyl-3,7-dihydro-purine-2,6-dione is described in U.S. Pat. No. 5,446,046.

The synthesis of the compounds of Formula II and III may be prepared by conventional methods known in the art. Specifically, these compounds can be prepared by methods taught in Suzuki et al., J. Med. Chem., 35, pp. 3581-3583 (1992) and Shimada et al., Tetrahedron Lett., 33, pp. 3151-3154 (1992).

The synthesis of compounds 7,8-dihydro-8-ethyl-2-(3-noradamantyl)-4-propyl-1H-imidazo[2,1-I]purine-5-(4H)-one; 8-(7-Hydroxy-3-noradamantyl)-1,3-dipropyl-3,7-dihydro-purine-2,6-dione; and 8-(3-noradamantyl)-1,3-dipropyl-3,7-dihydro-purine-2,6-dione is described in International Publication No. WO 95/31460 and its European counterpart application EP-619316 (1994).

The synthesis of compound 5-[8-(Isopropyl-methyl-amino)-9-methyl-9H-purin-6-ylamino]-bicyclo[2.2.1]heptan-2-ol is described in International Publication No. WO 96/06845 (1996).

The synthesis of compounds 1-[2-(2-Hydroxy-ethyl)-piperidin-1-yl]-3-(2-phenyl-pyrazolo[1,5-a]pyridin-3-yl)-propenone; 4-[6-Oxo-3-(2-phenyl-pyrazolo[1,5-a]pyridin-3-yl)-6H-pyridazin-1-yl]-butyric acid; and 6-(2-Phenyl-pyrazolo[1,5-a]pyridin-3-yl)-2-[2-(1H-tetrazol-5-yl)-ethyl]-2H-pyridazin-3-one is described in International Publication Nos. WO 95/18128 (1995), WO 96/33715 (1996), and WO 98/41237 (1998).

8-Cyclopentyl-1,3-dipropyl-3,7-dihydro-purine-2,6-dione (DPCPX) is commercially available from Research Biochemicals International;

The synthesis of 8-(3-Oxo-cyclopentyl)-1,3-dipropyl-3,7-dihydro-purine-2,6-dione (Apaxifylline) is described in International Publication No. WO 94/09787;

The synthesis of 8-(1-Amino-cyclopentyl)-1,3-dipropyl-3,7-dihydro-purine-2,6-dione is described in Ceccarelli, S. et al. Res. Commun. Mol. Pathol. Pharmacol., 87, pp. 101-102 (1995).

The synthesis of 8-Dicyclopropylmethyl-1,3-dipropyl-3,7-dihydro-purine-2,6-dione is described in Shimada J.; Suzuki F. et al. J. Med. Chem., 34, pp. 466-469 (1991).

In some embodiments, the compounds may be in the form of an achiral compound, an optically active compound, a pure diastereomer, a mixture of diastereomers, a prodrug or a pharmacologically acceptable salt thereof.

Production of A₁ Adenosine Receptor Antibodies

The invention also encompasses the use of antibodies raised against the A₁ adenosine receptor, as antagonists of the receptor. Such antibodies block the ligand (e.g., adenosine) binding site on the A₁ adenosine receptor or prevent the ligand (e.g., adenosine) from binding to the receptor.

The A₁ adenosine receptor may be used to elicit polyclonal or monoclonal antibodies which bind to the A₁ adenosine receptor using a variety of techniques well known to those of skill in the art. Alternatively, peptides corresponding to specific regions of the A₁ adenosine receptor may be synthesized and used to create immunological reagents according to well known methods.

The human A₁ adenosine receptor has been cloned and the DNA sequence encoding the receptor as well as the protein sequence for the receptor have been identified (see, e.g., Libert et al. Biochem Biophys Res Commun, 187(2), pp. 919-926 (1992); Townsend-Nicholson et al., Brain Res Mol Brain Res, 16(3-4), pp. 365-370 (1992)).

Antibodies directed against the A₁ adenosine receptor of this invention are immunoglobulin molecules or portions thereof that are immunologically reactive with the A₁ adenosine receptor of the present invention. More preferably, the antibodies used in the methods of the invention are immunologically reactive with the ligand binding domain of the A₁ adenosine receptor.

Antibodies directed against the A₁ adenosine receptor may be generated by immunization of a suitable host. Such antibodies may be polyclonal or monoclonal. Preferably they are monoclonal. Production of polyclonal and monoclonal antibodies is within ordinary skill in the art. For a review of methods useful in practicing the invention, see, e.g., Harlow and Lane (1988), Antibodies, A Laboratory Manual, Yelton, D. E. et al. (1981); Ann. Rev. of Biochem., 50, pp. 657-80., and Ausubel et al. (1989); Current Protocols in Molecular Biology (New York: John Wiley & Sons), updated annually. Determination of immunoreactivity with an A₁ adenosine receptor may be made by any of several methods well known in the art, including, e.g., immunoblot assay and ELISA.

Monoclonal antibodies with affinities of 10⁻⁸ M⁻¹ or preferably 10⁻⁹ to 10⁻¹⁰ M⁻¹ or stronger are typically made by standard procedures as described, e.g., in Harlow and Lane, (1988) supra. Briefly, appropriate animals are selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic A₁ adenosine receptor, or alternatively, to selection of libraries of antibodies in phage or similar vectors. See Huse et al., Science, 246, pp. 1275-81 (1989). Antibodies useful in the present invention may be employed with or without modification. Antigens (in this case the A₁ adenosine receptor) and antibodies can be labeled by joining, either covalently or non-covalently, a substance which provides for a detectable signal. Various labels and conjugation techniques are known in the art and can be employed in practicing the invention. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

An antibody of this invention may also be a hybrid molecule formed from immunoglobulin sequences from different species (e.g., mouse and human) or from portions of immunoglobulin light and heavy chain sequences from the same species. An antibody may be a single-chain antibody or a humanized antibody. It may be a molecule that has multiple binding specificities, such as a bifunctional antibody prepared by any one of a number of techniques known to those of skill in the art including the production of hybrid hybridomas, disulfide exchange, chemical cross-linking, addition of peptide linkers between two monoclonal antibodies, the introduction of two sets of immunoglobulin heavy and light chains into a particular cell line, and so forth.

The antibodies of this invention may also be human monoclonal antibodies, for example those produced by immortalized human cells, by SCID-hu mice or other non-human animals capable of producing “human” antibodies, or by the expression of cloned human immunoglobulin genes. The preparation of humanized antibodies is taught by U.S. Pat. Nos. 5,777,085 and 5,789,554.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

Uses for A₁ Adenosine Receptor Antagonists

The methods and compositions of this invention may be used to treat pulmonary diseases. The pulmonary disease can be, for example, pulmonary edema or pulmonary hypertension. These diseases may be caused by a variety of physical traumas.

In some embodiments of the present invention, the methods and compositions are used in the treatment of a pulmonary disease characterized by at least one condition selected from the group consisting of global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.

As used herein, “rales” and “crackles” mean abnormal sounds heard accompanying the normal respiratory sounds on auscultation of the chest.

The methods of this invention may be used to treat pulmonary edema caused by variety of conditions. These include but are not limited to an imbalance of Starling forces, altered alveolar-capillary membrane permeability (acute respiratory distress syndrome), lymphatic insufficiency. Moreover, pulmonary edema may be caused by a number of other conditions, including high-altitude pulmonary edema, neurogenic pulmonary edema, narcotic overdose, pulmonary embolism, eclampsia, after cardioversion, after anesthesia, after cardiopulmonary bypass.

The methods of this invention may be used to treat pulmonary edema caused by the imbalance of Starling forces. Causes for the imbalance of Starling forces include increased pulmonary capillary pressure, decreased plasma oncotic pressure due to hypoalbuminemia and increased negativity of interstitial pressure. Increased pulmonary capillary pressure has both cardiac and non-cardiac causes. The cardiac causes include left ventricular failure, mitral stenosis or subacute bacterial endocarditis. Non-cardiac causes include pulmonary venous fibrosis, congenital stenosis of the origin of the pulmonary veins or pulmonary venoocclusive disease. Increased pulmonary capillary pressure may also be caused by overperfusion of fluids.

The methods of this invention may be used to treat pulmonary edema caused by increased negativity of interstitial pressure. The causes of increased negativity of interstitial pressure include the rapid removal of the pneumothorax with large applied negative pressures or asthma.

The methods of this invention may be used to treat pulmonary edema caused by altered alveolar-capillary membrane permeability. The causes of altered alveolar-capillary membrane permeability include infectious pneumonia (viral or bacterial), inhaled toxins, circulating toxins, vasoactive substances (e.g., histamine, kinins), disseminated intravascular coagulation, immunologic reactions, radiation pneumonia, uremia, near drowning, aspiration pneumonia, smoke inhalation, adult respiratory distress syndrome.

The methods of this invention may be used to treat pulmonary edema caused by lymphatic insufficiency. The causes of lymphatic insufficiency include post-lung transplant insufficiency, lymphangitic carcinomatosis or fibrosing lymphangitis.

The methods of this invention may be used to treat pulmonary hypertension caused by variety of conditions. These include pulmonary arterial hypertension, pulmonary hypertension associated with disorders of the respiratory system and/or hypoxemia, pulmonary venous hypertension, pulmonary hypertension resulting from chronic thrombotic and/or embolic disease, pulmonary hypertension resulting from disorders directly affecting the pulmonary vasculature.

The methods of this invention may be used to treat pulmonary hypertension caused by pulmonary arterial hypertension. The causes of include primary pulmonary hypertension (including sporadic and familial disorders); related conditions such as collagen vascular disease, congenital systemic-to-pulmonary shunt, portal hypertension, and human immunodeficiency virus infection; drug and toxin induced (i.e., anorectic agents (appetite suppressants)); and persistent pulmonary hypertension of the newborn.

The methods of this invention may be used to treat pulmonary hypertension caused by pulmonary hypertension associated with disorders of the respiratory system and/or hypoxemia. The causes of pulmonary hypertension associated with disorders of the respiratory system and/or hypoxemia include chronic obstructive pulmonary disease, interstitial lung disease, sleep-disordered breathing, alveolar hypoventilation disorders, chronic exposure to high altitudes, neonatal lung disease and alveolar-capillary dysplasia.

The methods of this invention may be used to treat pulmonary hypertension caused by pulmonary venous hypertension. The causes of pulmonary venous hypertension include left-sided atrial or ventricular heart disease, left-sided valvular heart disease extrinsic compression of central pulmonary veins (e.g., fibrosing mediastinitis, adenopathy and/or tumors) and pulmonary veno-occlusive disease.

The methods of this invention may be used to treat pulmonary hypertension caused by chronic thrombotic and/or embolic disease. The causes of pulmonary hypertension resulting from chronic thrombotic and/or embolic disease include thromboembolic obstruction of proximal pulmonary arteries, obstruction of distal pulmonary arteries (e.g., pulmonary embolism (thrombus, tumor, ova and/or parasites, foreign material), in-situ thrombosis, sickle cell disease).

The methods of this invention may be used to treat pulmonary hypertension caused by disorders directly affecting the pulmonary vasculature. The causes of pulmonary hypertension resulting from disorders directly affecting the pulmonary vasculature include inflammatory conditions (e.g., schistosomiasis, sarcoidosis) and pulmonary capillary hemangiomatosis.

Pharmaceutical Compositions

The A₁ adenosine receptor antagonists may be formulated into pharmaceutical compositions for administration to animals, including humans. These pharmaceutical compositions, preferably include an amount of A₁ adenosine receptor antagonist effective to reduce vasoconstriction or enhance pulmonary hemodynamics and a pharmaceutically acceptable carrier.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single-bolus dose, an infusion or a loading bolus dose followed with a maintenance dose. These compositions may be administered once a day or on an “as needed” basis.

The pharmaceutical compositions of this invention may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Topical application can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of A₁ adenosine receptor antagonist that may be combined with the carrier materials to produce a single dosage form will vary depending upon the host treated and the particular mode of administration. The compositions can be formulated so that a dosage of between 0.01-100 mg/kg body weight of the A₁ adenosine receptor antagonist is administered to a patient receiving these compositions. In some ebodiments of the invention, the dosage is 0.1-10 mg/kg body weight. The composition may be administered as a single dose, multiple doses or over an established period of time in an infusion.

A specific dosage and treatment regimen for any particular patient will depend upon a variety of factors, including the particular A₁ adenosine receptor antagonist, the patient's age, body weight, general health, sex, and diet, and the time of administration, rate of excretion, drug combination, and the severity of the particular disease being treated. Judgment of such factors by medical caregivers is within ordinary skill in the art. The amount of antagonist will also depend on the individual patient to be treated, the route of administration, the type of formulation, the characteristics of the compound used, the severity of the disease, and the desired effect. The amounts of antagonists can be determined by pharmacological and pharmacokinetic principles well-known in the art.

According to some embodiments, the invention provides methods for reducing pulmonary vasoconstriction or improving pulmonary hemodynamics comprising the step of administering to a patient one of the above-described pharmaceutical compositions. The term “patient”, as used herein, means an animal, e.g., a human.

In order that the invention described herein may be more fully understood, the following examples are set forth. It should be understood that these examples are for illustrative purposes only and are not to be construed as limiting this invention in any manner.

### EXAMPLE 1

Animal Model

Nineteen Yorkshire pigs (20-25 kg, male Hambone Farms, SC) were instrumented in order to induce pacing cardiac heart failure as described in Tomita et al., Circulation, 83, pp. 635-644 (1991). Briefly, under isoflurane anesthesia (3% in 1.5 L/min of oxygen) and through a left thoracotomy, a shielded stimulated electrode was sutured onto the left atrium, connected to a modified programmable pacemaker (8329 Medtronic, Inc., Minneapolis, Minn.) and buried in a subcutaneous pocket. Ten to fourteen days following recovery from the surgical procedure, a baseline echocardiographic study was performed and pacing initiate at 240 beats/min for 3 weeks. An additional group of 7 normal control animals were cared for in identical fashion with the exception of the pacing protocol. At the conclusion of the 3-week pacing period, the pacemakers were de-activated and echocardiographic studies were performed. For these studies, the animals were brought to the laboratory and the pacemakers were deactivated. Two-dimensional and M-mode echocardiographic studies (ATL Ulmark VI, 2.25 MHz transducer, Bothell, Wash.) were used to image the left ventricle from a right parasternal approach. Following the echocardiographic study, the animals were prepared for acute instrumentation and initiation of the study protocol.

Acute Instrumentation

The pigs were anesthetized with intravenous boluses of sufentanyl 2.0 μg/kg, etomidate 0.3 mg/kg, and vecuronium 10 mg, after which a tracheostomy was performed. A tubocurarine 12 mg intravenous bolus was administered after obtaining arterial pressure. Anesthesia was maintained throughout the procedure by continuous intravenous infusions of morphine sulfate 3 mg/kg/hr and tubocurarine 2 mg/hr. Etomidate 0.1 mg/kg intravenous was also given at 30 minute intervals. A maintenance infusion of 10 ml/kg/hr of lactated Ringer's solution was maintained throughout the protocol. This anesthetic protocol resulted in a deep anesthetic plane and stable hemodynamic profiles for up to 6 hours. A multi-lumened thermodilution catheter (7.5 Fr, Baxter Healthcare Corp., Irvine, Calif.) was positioned in a pulmonary artery via the right external jugular vein and a large bore catheter (7 Fr) was placed in the left external jugular vein for fluid administration. The carotid artery was exposed and cannulated, and the catheter (7 Fr) was advanced to the aortic root for aortic blood pressure measurements and blood samplings.

Hemodynamic and Renal Function Measurements

Following instrumentation and a 15-minute stabilization period, baseline hemodynamics were recorded and digitized. Thermodilution derived cardiac output and ejection fraction were obtained from the pulmonary artery catheter in triplicate. All measurements were simultaneously recorded with the ventilator temporarily suspended in order to prevent respiratory artifact the recordings. An arterial sample was drawn for electrolyte assays. Pulmonary and systemic vascular resistances were computed from the thermodilution cardiac output and pressure measurements using standard formulae.

Experimental Protocol

Following instrumentation and collection of baseline measurements, the animals were randomly assigned to receive either vehicle infusion (polyethylene glycol, 3 ml intravenous, n=10) or the A₁ adenosine receptor antagonist (1 mg/kg 1,8-(3-Oxa-tricyclo[3.2.1.0^{2,4}]oct-6-yl)-1,3-dipropyl-3,7-dihydro-purine-2,6-dione (BG9719); n=9). Following infusion of vehicle or BG9719, the hemodynamic measurements described in the previous section were repeated at 10, 30, 60, 90, 120 minutes post infusion.

Data Analysis

Changes in hemodynamics were initially examined between the control and A₁ adenosine receptor antagonist (BG9719) groups by ANOVA. Comparisons between these baseline values following randomization were performed by a 2-way ANOVA. Comparisons of these parameters following infusion were compared using a multi-way ANOVA for repeated measures. Pair-wise comparisons were performed with a Bonferroni adjusted t-test. All statistical analyses were performed using statistical software programs (BMDP Statistical Software Inc. University of California Press, Los Angeles, Calif.). Results were as mean±standard error of the mean (SEM). Values of p<0.05 were considered to be statistically significant.

Systemic and Pulmonary Hemodynamics in Heart Failure

In the pacing heart failure group, left ventricular end diastolic dimension increased (5.68±0.15 vs. 4.09±0.12 cm; p<0.05) and fractional shortening decreased (24±2 vs. 42±2%; p, 0.05) compared to normal control values. In the heart failure group, heart rate, pulmonary artery pressure and pulmonary capillary wedge pressure were increased and cardiac output and mean aortic pressure reduced when compared to normal control values. There were no differences in any of the baseline parameters in those animals randomly assigned for A₁ adenosine receptor antagonist or vehicle infusions. No change from baseline in hemodynamic measurements was noted in the normal control group throughout the study.

Systemic and Pulmonary Hemodynamics—Effects of A₁ Adenosine Receptor Antagonists in Heart Failure

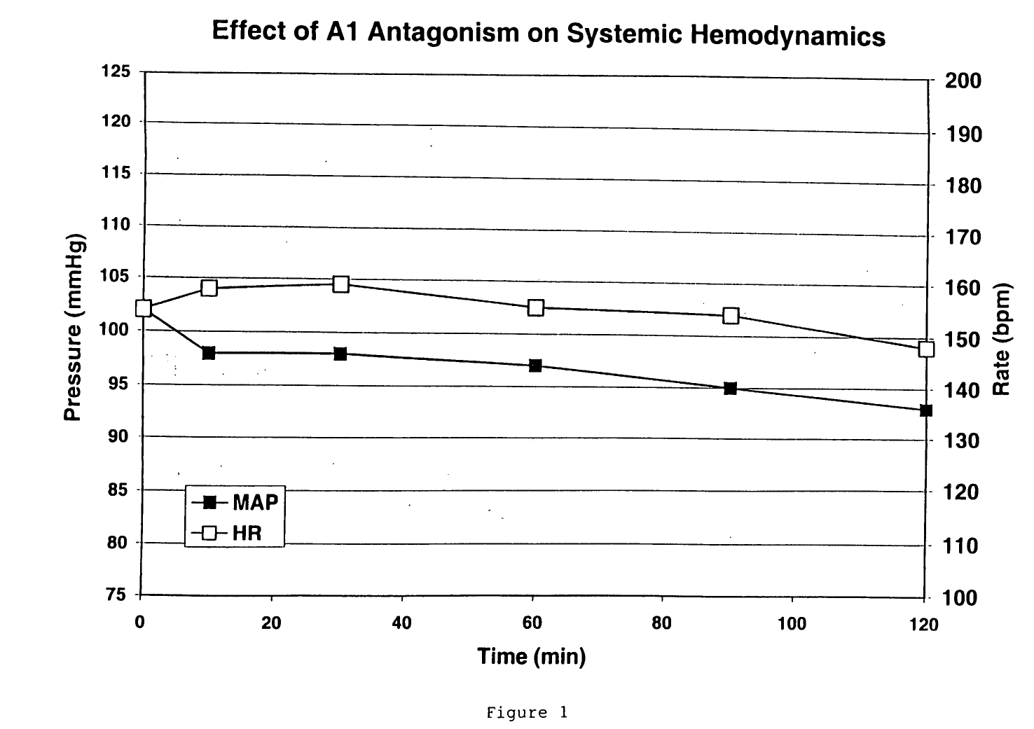

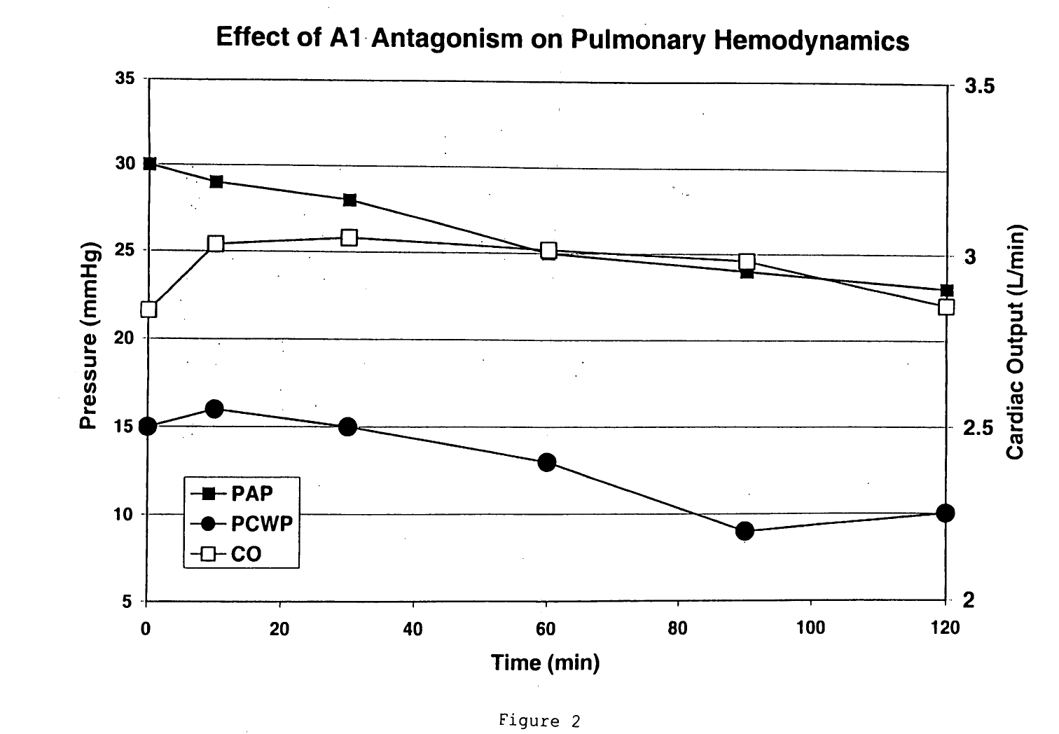

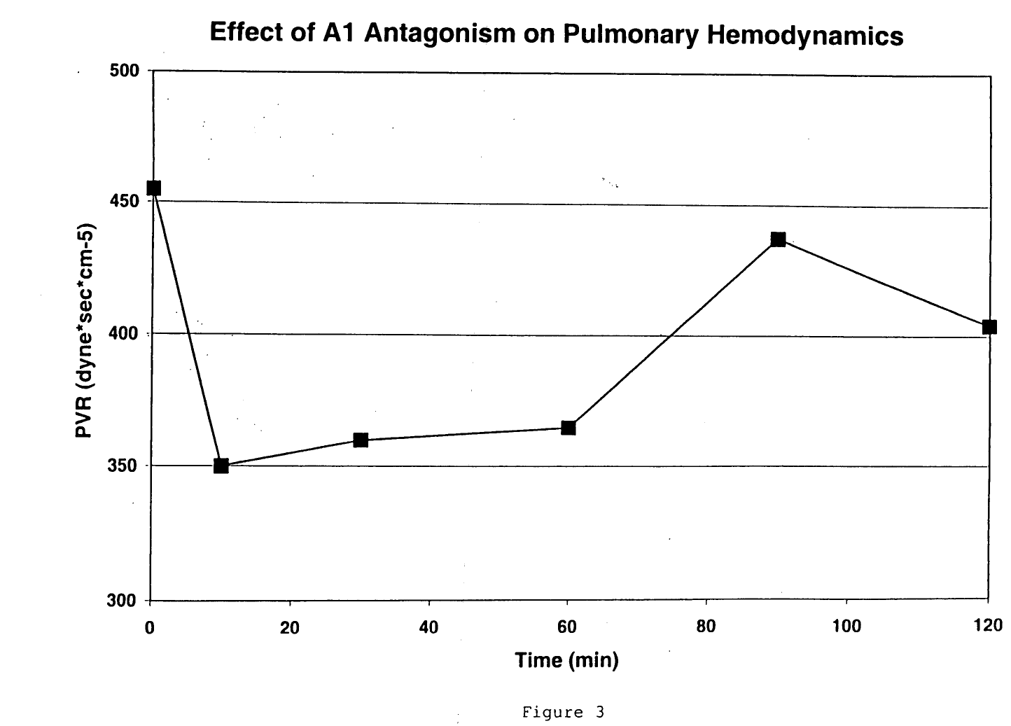

No change from baseline in heart rate (FIG. 1), mean arterial pressure (FIG. 1), cardiac output (FIG. 2), or systemic vascular resistance was noted following treatment with A₁ adenosine receptor antagonist BG9719. Mean pulmonary artery pressure fell from baseline at 30 min post treatment with BG9719 and remained depressed (30±1 vs. 23±3 mmHg; p<0.05) (FIG. 2). Pulmonary capillary wedge pressure (PCWP) decreased at 90 minutes post treatment with BG9719 (9±2 mg Hg; p<0.05) (FIG. 2). Pulmonary vascular resistance fell by 38% from baseline at 10 min post treatment with BG9719 and returned to baseline levels (FIG. 3). In the vehicle group, no changes in hemodynamics were noted. Selective A₁ adenosine receptor antagonism with BG9719 was associated with an acute decrease in pulmonary resistive properties without reducing systemic vascular tone or blood pressure.

### EXAMPLE 2

Animal Model

Four Yorkshire pigs (25-30 kg, male, Hambone Farms, SC) were implanted with a pacemaker (8329, Medtronic, Inc., Minneapolis, Minn.) in order to induce pacing CHF as described above. Ten to 14 days following recovery from the surgical procedure, a baseline echocardiographic study (ATL Ultramark VI, 2.25 MHz transducer, Bothell, Wash.) was performed and pacing initiated at 240 beats/min for 3 weeks. An additional group of 6 normal control animals were cared for in identical fashion with the exception of the pacing protocol. At the conclusion of the 3-week pacing period, the pacemakers were deactivated and echocardiographic studies were used to image the LV from a right parasternal approach. Following the echocardiographic study, the animals were prepared for acute instrumentation and initiation of the study protocol.

Acute Instrumentation

The pigs were anesthetized (i.v. sufentanyl 2.0 g/kg, etomidate 0.3 mg/kg) and paralyzed (vecuronium 10 mg, tubocurarine 12 mg). A maintenance infusion of 10 ml/kg/hr of lactated ringer's solution was maintained throughout the protocol. A thermodilution catheter (7.5 Fr, Baxter Healthcare Corp., Irvine, Calif.) was positioned in the pulmonary artery via the right external jugular vein and a large bore catheter (7 Fr) was placed in the left external jugular vein for fluid administration. The carotid artery was exposed and cannulated, and the catheter (7 Fr) was advanced to the aortic root for aortic blood pressure measurements and blood sampling drained.

Hemodynamic Function Measurements

Following instrumentation and a 10-minute stabilization period, baseline hemodynamics were recorded and digitized. Thermodilution derived cardiac output and ejection fraction were obtained from the pulmonary artery catheter in triplicate. Pulmonary and systemic vascular resistances were computed from the pressure measurements and cardiac output and using standard formulae.

Experimental Protocol

Following instrumentation and collection of baseline measurements, the A₁ receptor antagonist (3-[4-(2,6-Dioxo-1,3-dipropyl-2,3,6,7-tetrahydro-1H-purin-8-yl)-bicyclo[2.2.2]oct-1-yl]-propionic acid (BG9928), 1 mg/kg) was infused intravenously. Hemodynamic measurements described in the previous section were repeated at 10, 20, 30, 60, 90, and 120 minutes post infusion.

Data Analysis

Comparisons of basal hemodynamics between the control and HF groups were performed using Student's t-test. Time-dependent changes in hemodynamics following A₁ block infusion were examined by ANOVA. Pair wise comparisons were performed with a Bonferroni adjusted t-test. All statistical analyses were performed using statistical software programs (BMDP Statistical Software Inc. University of California Press, Los Angeles, Calif.). Results are presented as mean ± standard error of the mean (SEM). Values of p<0.05 were considered to be statistically significant.

Results

In the pacing heart failure group, left ventricular end-diastolic dimension increased (5.8±0.1 vs. 4.1±0.3 cm; p<0.05) and fractional shortening decreased (20±1 vs. 41±2%; p<0.05) compared to normal control values. Baseline left ventricular function and hemodynamics are summarized in Table 1. In the heart failure group, heart rate, pulmonary artery pressure, and pulmonary capillary wedge pressure (PCWP) were increased, and stroke volume was reduced when compared to normal control values. Pulmonary vascular resistances were also elevated in the HF group compared to normal. No change from baseline in hemodynamics was noted in the normal control group throughout the study.

Systemic and Pulmonary Hemodynamics: Effects of A₁ Adenosine Receptor Antagonist in Heart Failure

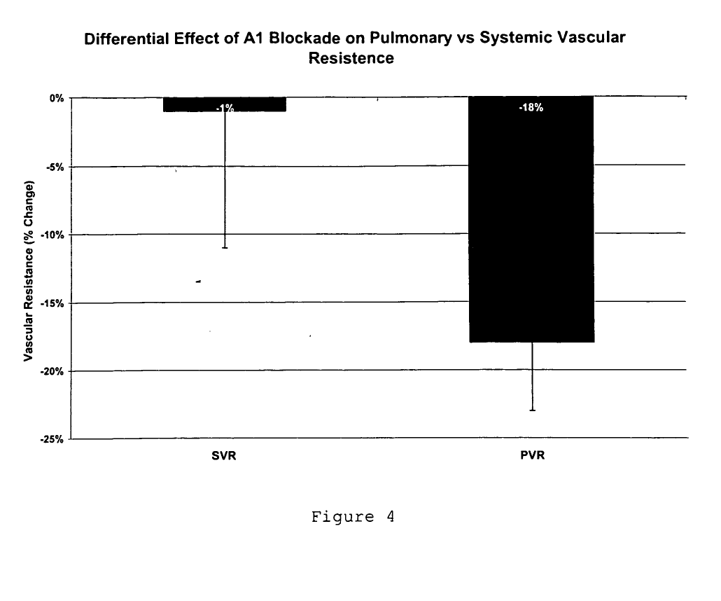

No change from baseline in heart rate, mean arterial pressure, or cardiac output was noted following treatment with the A₁ adenosine antagonist BG9928. Pulmonary vascular resistance fell by 18% from baseline at 10 min post treatment with BG9928 (p<0.05) while there was no change in systemic vascular resistance (FIG. 4). Selective A₁ adenosine receptor antagonism with BG9228 was associated with an acute decrease in pulmonary resistive properties without reducing systemic vascular tone or blood pressure.

TABLE 1
Baseline LV Function and Hemodynamics
in Normal vs. CHF preparations

| | Control | Pacing CHF | p-values |
|---|---|---|---|
| LV Function | | | |
| Fractional Shortening (%) | 41 ± 2 | 20 ± 1* | <0.01 |
| End Diastolic Dimension (cm) | 4.1 ± 0.3 | 5.8 ± 0.1* | <0.01 |
| Hemodynamics | | | |
| Heart Rate (bpm) | 94 ± 3 | 126 ± 17 | 0.16 |
| Mean Arterial Pressure (mmHg) | 103 ± 8 | 92 ± 3 | 0.15 |
| Mean PA Pressure (mmHg) | 14 ± 3 | 28 ± 4 | 0.03 |
| PCWP (mmHg) | 6 ± 2 | 14 ± 2 | 0.04 |
| Cardiac Output (L/min) | 4.15 ± 0.25 | 3.54 ± 0.31 | 0.08 |
| Stroke Volume (mL) | 44.4 ± 3.3 | 29.5 ± 3.8 | 0.02 |
| Cardiac Index (L/min/kg) | 0.13 ± 0.01 | 0.09 ± 0.01 | 0.03 |
| Stroke Volume Index (mL/kg) | 1.41 ± 0.09 | 0.76 ± 0.09 | <0.01 |
| Resistance | | | |
| Systemic (dyne.s.cm-5) | 2036 ± 227 | 2107 ± 148 | 0.80 |
| Pulmonary (dyne.s.cm-5) | 148 ± 29 | 328 ± 50 | 0.04 |
| Systemic Indexed (x₁02 | 645 ± 85 | 823 ± 81 | 0.07 |
| dyne.s.cm-5.kg) | | | |
| Pulmonary (x₁02 dyne.s.cm-5.kg) | 47 ± 10 | 126 ± 18 | <0.01 |
| Sample Size (n) | 6 | 4 | |

Values presented as Mean ± SEM. p-values for comparison between control and CHF as indicated
CHF: rapid pacing at 240 bpm for 3 weeks.
PCWP: Pulmonary Capillary Wedge Pressure

### EXAMPLE 3

Evaluation of A₁Selective Antagonists—Inhibition of Adenosine-Mediated Vasoconstriction of Pulmonary Vessels.

To evaluate a larger number of compounds, a model was designed wherein pulmonary vessels from rodents (rats) are obtained and transverse rings of the vessel are used in an in vitro tissue bath apparatus. This model allows for evaluation of compounds to determine if the test compounds reduce pulmonary vasoconstriction.

Male Sprague Dawley rats are anesthetized IP with 90 mg/kg of Brevital sodium. After achieving a surgical plane of anesthesia, the thoracic area is shaved and the heart and thoracic region are exposed by a median sternotomy. The pulmonary artery is harvested by removing the esophagus, resecting the trachea, and exposing the major blood vessels entering the dorsal surface of the heart. The pulmonary artery is gently dissected and removed. The isolated vessel is kept in an open container of cold Krebs-Henseleit buffer, pH 7.4 containing D-glucose (2 g/l), MgSO₄ (0.14 g/l), potassium sulfate monobasic (0.16 g/l), KCl (0.35 g/l), NaCl (6.9 g/l), CaCl (0.373 g/l), and Na bicarbonate (2.1 g/l) until it is ready to use. Using a petri plate and a stereomicroscope, the vessel is cleaned of adventitia and cut into 3 mm ring segments. The pulmonary rings are then mounted carefully onto wire triangles, and placed in pre-heated 37° C. organ baths containing 10 mls of Krebs-Henseleit buffer bubbled with 95% O2/5% CO₂. Two lengths of 3-0 silk thread with triangular wire supports at each end is used to support pulmonary rings; one end of the assembly is hooked up to an L-shaped glass rod and the other end to an isometric force transducer to measure force in gram tension. Manual preload tension is set at 1 g and rings are allowed to equilibrate for 1 hour, with washing and preload adjustment every 15 minutes or as needed. Following equilibration, pulmonary rings are challenged with 60 mM Potassium Chloride (KCl) and allowed to plateau up to 5 minutes and washed.

The reactivity of the vessels is tested by application of PGF₂ₐ, phenylephrine, or potassium. After the reactivity is confirmed, the tissue is washed three times and allowed to stabilize under 1 g tension. A concentration-response curve is then obtained with the A₁ selective agonist N-6 cyclopentyl adenosine (CPA) while under oxygenation. The tissue is then washed three times and allowed to equilibrate under 1 gm tension without oxygenation and with incubation with various concentrations of the test antagonist. The CPA concentration-response curve is then repeated to confirm vasoconstrictive response under hypoxia and to determine if the antagonist causes a rightward, parallel shift in the agonist concentration-response curve (indicating full, competitive antagonism).

Throughout this specification and claims, the word “comprise”, or variations such as “comprises” or “comprising”, will be understood to imply the inclusion of a stated integer or group of integers but not the exclusion of any other integer or groups of integers.

While we have hereinbefore presented a number of embodiments of this invention, it is apparent that the these embodiments can be altered to provide other embodiments of this invention. Therefore, it will be appreciated that the scope of this invention is not limited to the specific embodiments presented by way of example.

1. A method for reducing pulmonary vasoconstriction or improving pulmonary hemodynamics in a patient comprising administering to the patient a pharmaceutically effective amount of an A₁ adenosine receptor antagonist.

2. The method of claim 1, wherein the adenosine A receptor antagonist is selected from the group consisting of:
a. a compound comprising the formula I: 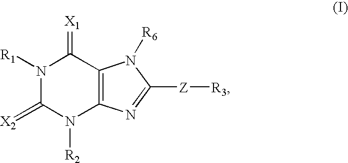
wherein R₁ and R₂ are independently selected from the group consisting of:
1) hydrogen;
2) alkyl, alkenyl of not less than 3 carbons, or alkynyl of not less than 3 carbons; wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and
3) aryl or substituted aryl;
R₃ is selected from the group consisting of:
1) a bicyclic, tricyclic or pentacyclic group selected from the group consisting of: 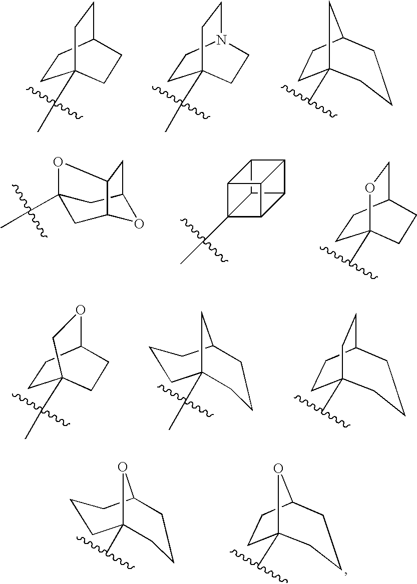
wherein the bicyclic or tricyclic group is either unsubstituted or functionalized with one or more substitents selected from the group consisting of:
a) alkyl, alkenyl, and alkynyl; wherein each alkyl, alkenyl, or alkynyl group is either unsubstituted or functionalized with one or more substituents selected from the group consisting of (amino) (R₅)acylhydrazinylcarbonyl, (amino)(R₅)acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and
b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, hydroxy, oximino, phosphate, substituted aralkylamino, substituted heterocyclyl, substituted heterocyclylsulfonylamino, sulfoxyacylamino, and thiocarbamoyl; and
2) the tricyclic group: 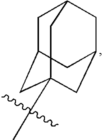
wherein the tricyclic group is functionalized with one or more substituents selected from the group consisting of:
a) alkyl, alkenyl, and alkynyl; wherein each alkyl, alkenyl, or alkynyl group is either unsubstituted or functionalized with one or more substituents selected from the group consisting of (amino)(R₅)acylhydrazinylcarbonyl, (amino) (R₅) acyloxycarboxy, (hydroxy)(carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R₅, R₅-alkoxy, R₅-alkylamino, cyano, cyanoalkylcarbamoyl, cycloalkylamino, dialkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and
b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, cyano, cycloalkylamino, dialkylaminoalkylcarbamoyl, halogen, heterocyclyl, heterocyclylalkylamino, oximino, phosphate, substituted aralkylamino, substituted heterocyclyl, substituted heterocyclylsulfonylamino, sulfoxyacylamino, and thiocarbamoyl;
3) a bicyclic or tricyclic group selected from the group consisting of: 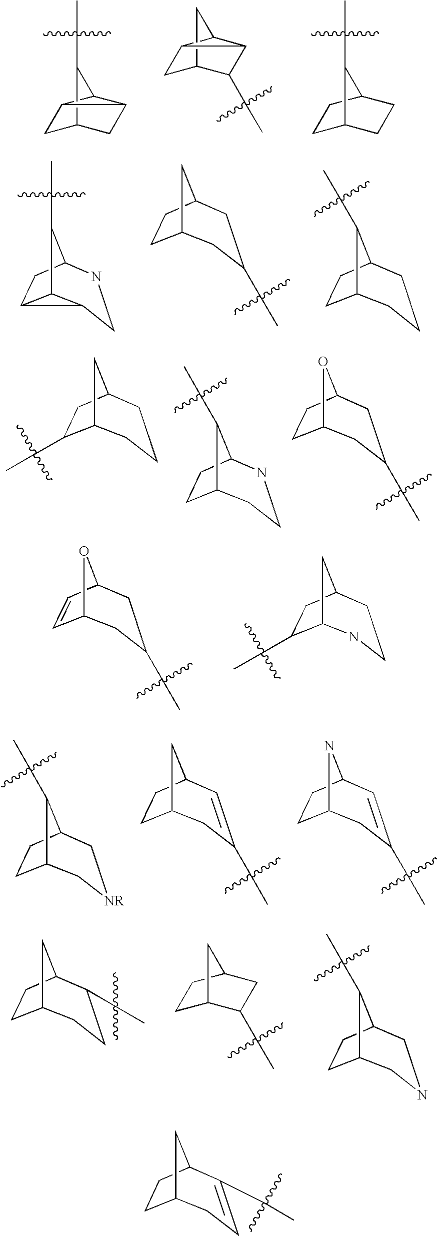
wherein the bicyclic or tricyclic group is either unsubstituted or fuctionalized with one or more substituents selected from the group consisting of:
a) alkyl, alkenyl, and alkynyl; wherein the alkyl, alkenyl, and alkynyl are either unsubstituted or functionalized with one or more substituents selected from the group consisting of alkoxy, alkoxycarbonyl, alkoxycarbonylaminoalkylamino, aralkoxycarbonyl, —R₅, dialkylamino, heterocyclylalkylamino, hydroxy, substituted arylsulfonylaminoalkylamino, and substituted heterocyclylaminoalkylamino;
b) acylaminoalkylamino, alkenylamino, alkoxycarbonyl, alkoxycarbonylalkylamino, alkoxycarbonylaminoacyloxy, alkoxycarbonylaminoalkylamino, alkylamino, amino, aminoacyloxy, carbonyl, —R₅, R₅-alkoxy, R₅-alkylamino, dialkylaminoalkylamino, heterocyclyl, heterocyclylalkylamino, hydroxy, phosphate, substituted arylsulfonylaminoalkylamino, substituted heterocyclyl, and sustituted heterocyclylaminoalkylamino;
R₄ is selected from the group consisting of hydrogen, C₁₋₄-alkyl, C₁₋₄-alkyl-CO₂H, and phenyl, wherein the C₁₋₄-alkyl, —C₁₋₄-alkyl-CO₂H, and phenyl groups are either unsubstituted or functionalized with one to three substituents selected from the group consisting of halogen, —OH, —OMe, —NH₂, NO₂, benzyl, and benzyl functionalized with one to three substituents selected from the group consisting of halogen, —OH, —OMe, —NH₂, and —NO₂;
R₅ is selected from the group consisting of —CH₂COOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following: 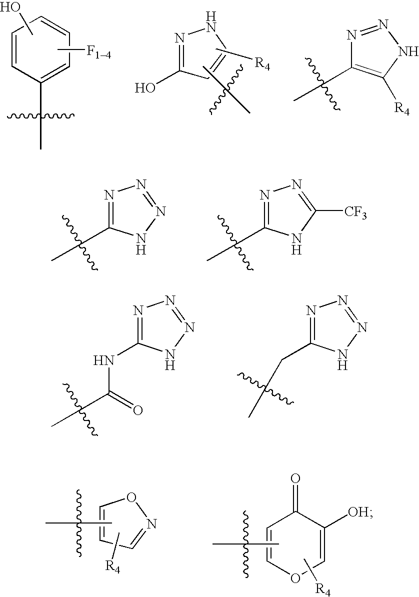
X₁ and X₂ are independently selected from the group consisting of O and S;
Z is selected from the group consisting of a single bond, —O—, —(CH₂)₁₋₃—, —O(CH₂)₁₋₂—, —CH₂OCH₂—, —(CH₂)₁₋₂O—, —CH═CHCH₂—, —CH═CH—, and —CH₂CH═CH—; and
R₆ is selected from the group consisting of hydrogen, alkyl, acyl, alkylsulfonyl, aralkyl, substituted aralkyl, substituted alkyl, and heterocycle; and
b. a compound of formula II or III: 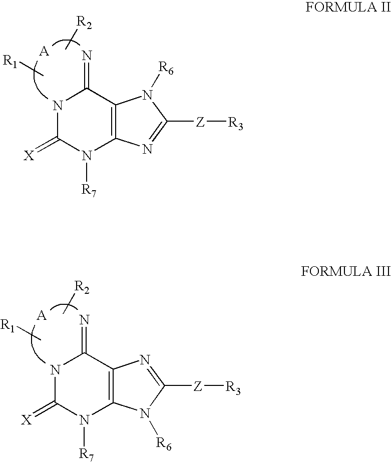
wherein R₁ and R₂ are independently selected from the group consisting of:
1) hydrogen;
2) alkyl, alkenyl or alkynyl, wherein said alkyl, alkenyl, or alkynyl is either unsubstituted or functionalized with one or more substituents selected from the group consisting of hydroxy, alkoxy, amino, alkylamino, dialkylamino, heterocyclyl, acylamino, alkylsulfonylamino, and heterocyclylcarbonylamino; and
3) aryl or substituted aryl;
R₃ is selected from the group consisting of:
1) a bicyclic, tricyclic or pentacyclic group selected from the group consisting of: 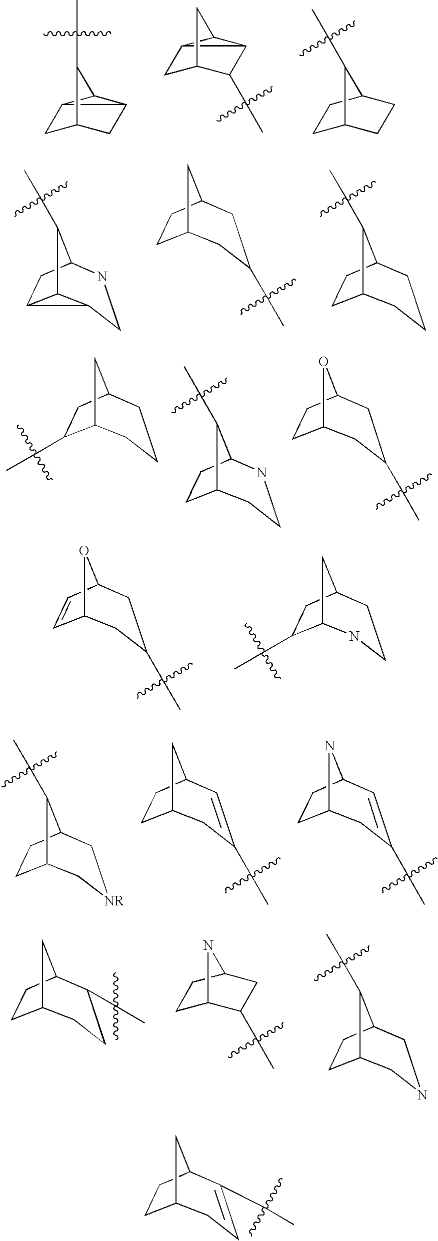 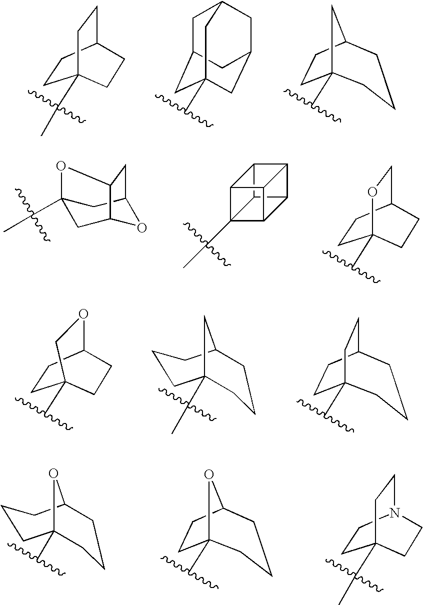
wherein the bicyclic, tricyclic or pentacyclic group is either unsubstituted or functionalized with one or more substituents selected from the group consisting of:
i) alkyl, alkenyl and alkynyl; wherein each alkyl, alkenyl or alkynyl group is either unsubstituted or functionalized with one or more substituents selected from the group consisting of (alkoxycarbonyl)aralkylcarbamoyl, (amino)(R₅)acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl; and
ii) (alkoxycarbonyl)aralkylcarbamoyl, (amino) (R₅) acylhydrazinylcarbonyl, (amino)(R₅)acyloxycarboxy, (hydroxy)(carboalkoxy)alkylcarbamoyl, acylaminoalkylamino, acyloxy, aldehydo, alkenoxy, alkenylamino, alkenylsulfonylamino, alkoxy, alkoxycarbonyl, alkoxycarbonylalkylamino, alkoxycarbonylamino, alkoxycarbonylaminoacyloxy, alkoxycarbonylaminoalkylamino, alkylamino, alkylaminoalkylamino, alkylcarbamoyl, alkylphosphono, alkylsulfonylamino, alkylsulfonyloxy, amino, aminoacyloxy, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonyl, aralkoxycarbonylamino, arylheterocyclyl, aryloxy, arylsulfonylamino, arylsulfonyloxy, carbamoyl, carbonyl, cyano, cyanoalkylcarbamoyl, cycloalkylamino, dialkylamino, dialkylaminoalkylamino, dialkylaminoalkylcarbamoyl, dialkylphosphono, haloalkylsulfonylamino, halogen, heterocyclyl, heterocyclylalkylamino, heterocyclylcarbamoyl, hydroxy, hydroxyalkylsulfonylamino, oximino, phosphate, phosphono, —R₅, R₅-alkoxy, R₅-alkyl(alkyl)amino, R₅-alkylalkylcarbamoyl, R₅-alkylamino, R₅-alkylcarbamoyl, R₅-alkylsulfonyl, R₅-alkylsulfonylamino, R₅-alkylthio, R₅-heterocyclylcarbonyl, substituted aralkylamino, substituted arylcarboxyalkoxycarbonyl, substituted arylsulfonylaminoalkylamino, substituted heteroarylsulfonylamino, substituted heterocyclyl, substituted heterocyclylaminoalkylamino, substituted heterocyclylsulfonylamino, sulfoxyacylamino, thiocarbamoyl, trifluoromethyl;
R₄ is selected from the group consisting of hydrogen, C₁₋₄-alkyl, C₁₋₄-alkyl-CO₂H, and phenyl, wherein the C₁₋₄-alkyl, C₁₋₄-alkyl-CO₂H, and phenyl groups are either unsubstituted or functionalized with one to three substituents selected from the group consisting of halogen, —OH, —OMe, —NH₂, NO₂, benzyl, and benzyl functionalized with one to three substituents selected from the group consisting of halogen, —OH, —OMe, —NH₂, and —NO₂;
R₅ is selected from the group consisting of —(CR₁R₂)ₙCOOH, —C(CF₃)₂OH, —CONHNHSO₂CF₃, —CONHOR₄, —CONHSO₂R₄, —CONHSO₂NHR₄, —C(OH)R₄PO₃H₂, —NHCOCF₃, —NHCONHSO₂R₄, —NHPO₃H₂, —NHSO₂R₄, —NHSO₂NHCOR₄, —OPO₃H₂, —OSO₃H, —PO(OH)R₄, —PO₃H₂, —SO₃H, —SO₂NHR₄, —SO₃NHCOR₄, —SO₃NHCONHCO₂R₄, and the following: 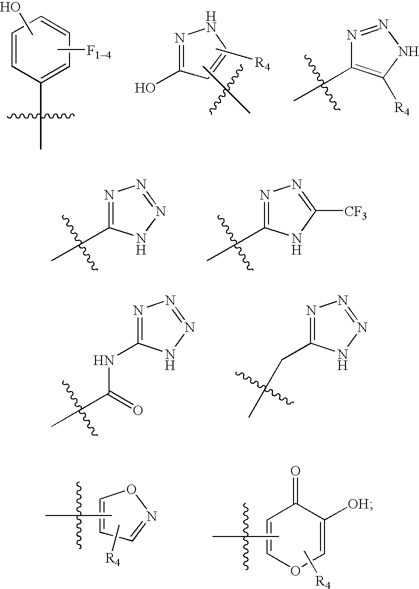
n=0, 1, 2 or 3;
A is selected from the group consisting of —CH═CH, —(CH)ₘ—(CH)_{m,}, CH═CH—CH₂, and —CH₂—CH═CH;
m=1 or 2;
X is O or S;
z is selected from the group consisting of a single bond, —O—, —(CH₂)ₙ—, —O(CH₂) 1-2%, —CH₂OCH₂—, —(CH₂)₁₋₂O—, —CH═CHCH₂—, —CH═CH—, and —CH₂CH═CH—; and
R₆ is selected from the group consisting of hydrogen, alkyl, acyl, alkylsufonyl, aralkyl, substituted aralkyl, substituted alkyl, and heterocyclyl; and
R₇ is selected from the group consisting of:
1) hydrogen;
2) alkyl, alkenyl of not less than 3 carbons, or alkynyl of not less than 3 carbons; wherein said alkyl, alkenyl or alkynyl is either unsubstituted or functionalized with one or more substitutents selected from the group consisting of hydroxy, alkoxy, amino, alkylamino, dialkylamino, heterocyclyl, acylamino, alkylsulfonylamino, and heterocyclylcarbonylamino; and
5) aryl or substituted aryl;
alkylaryl or alkyl substituted aryl;
c. 8-(3-Oxa-tricyclo[3.2.1.0^{2,4}]oct-6-yl)-1,3-dipropyl-3,7-dihydro-purine-2,6-dione;
8-Bicyclo[2.2.1]hept-5-en-2-yl-1,3-dipropyl-3,7-dihydro-purine-2,6-dione;
7,8-dihydro-8-ethyl-2-(3-noradamantyl)-4-propyl-1H-imidazo[2,1-I]purine-5-(4H)-one;
8-(7-Hydroxy-3-noradamantyl)-1,3-dipropyl-3,7-dihydro-purine-2,6-dione;
8-(3-noradamantyl)-1,3-dipropyl-3,7-dihydro-purine-2,6-dione;
5-[8-(Isopropyl-methyl-amino)-9-methyl-9H-purin-6-ylamino]-bicyclo[2.2.1]heptan-2-ol;
1-[2-(2-Hydroxy-ethyl)-piperidin-1-yl]-3-(2-phenyl-pyrazolo[1,5-a]pyridin-3-yl)-propenone;
4-[6-Oxo-3-(2-phenyl-pyrazolo[1,5-a]pyridin-3-yl)-6H-pyridazin-1-yl]-butyric acid;
6-(2-Phenyl-pyrazolo[1,5-a]pyridin-3-yl)-2-[2-(1H-tetrazol-5-yl)-ethyl]-2H-pyridazin-3-one;
8-Cyclopentyl-1,3-dipropyl-3,7-dihydro-purine-2,6-dione (DPCPX);
8-(3-Oxo-cyclopentyl)-1,3-dipropyl-3,7-dihydro-purine-2,6-dione (Apaxifylline);
8-(1-Amino-cyclopentyl)-1,3-dipropyl-3,7-dihydro-purine-2,6-dione; and
8-Dicyclopropylmethyl-1,3-dipropyl-3,7-dihydro-purine-2,6-dione.

3. The method of claim 1, wherein the A₁ adenosine receptor antagonist is selected from the group consisting of:
3-[4-(2,6-Dioxo-1,3-dipropyl-2,3,6,7-tetrahydro-1H-purin-8-yl)-bicyclo[2.2.2]oct-1-yl]-propionic acid;
8-(3-Oxa-tricyclo[3.2.1.0^{2,4}] oct-6-yl)-1,3-dipropyl-3,7-dihydro-purine-2,6-dione;
8-Bicyclo[2.2.1]hept-5-en-2-yl-1,3-dipropyl-3,7-dihydro-purine-2,6-dione;
7,8-Dihydro-8-isopropyl-2-(4-Hydroxy-bicyclo[2.2.2]oct-1-yl)-4-propyl-1H-imidazo[2,1-i]purin-5-(4H)-one;
7,8-Dihydro-8-ethyl-2-(4-Hydroxy-bicyclo[2.2.2]oct-1-yl)-4-propyl-1H-imidazo[2,1-i]purin-5-(4H)-one;
3-[4-(2,6-Dioxo-1,3-dipropyl-2,3,6,7-tetrahydro-1H-purin-8-yl)-bicyclo[2.2.2]oct-1-yloxy]-propionic acid;
8-(1-Hydroxy-tricyclo[2.2.1.0^{2,6}]hept-3-yl)-1,3-dipropyl-3,7-dihydro-purine-2,6-dione;
8-(4-Hydroxy-bicyclo[2.2.2]oct-1-yl)-1,3-dipropyl-3,7-dihydro-purine-2,6-dione;
7,8-dihydro-8-ethyl-2-(3-noradamantyl)-4-propyl-1H-imidazo[2,1-I]purine-5-(4H)-one;
8-(7-Hydroxy-3-noradamantyl)-1,3-dipropyl-3,7-dihydro-purine-2,6-dione;
8-(3-noradamantyl)-1,3-dipropyl-3,7-dihydro-purine-2,6-dione;
5-[8-(Isopropyl-methyl-amino)-9-methyl-9H-purin-6-ylamino]-bicyclo[2.2.1]heptan-2-ol;
1-[2-(2-Hydroxy-ethyl)-piperidin-1-yl]-3-(2-phenyl-pyrazolo[1,5-a]pyridin-3-yl)-propenone;
4-[6-Oxo-3-(2-phenyl-pyrazolo[1,5-a]pyridin-3-yl)-6H-pyridazin-1-yl]-butyric acid;
6-(2-Phenyl-pyrazolo[1,5-a]pyridin-3-yl)-2-[2-(1H-tetrazol-5-yl)-ethyl]-2H-pyridazin-3-one;
8-Cyclopentyl-1,3-dipropyl-3,7-dihydro-purine-2,6-dione (DPCPX);
8-(3-Oxo-cyclopentyl)-1,3-dipropyl-3,7-dihydro-purine-2,6-dione (Apaxifylline);
8-(1-Amino-cyclopentyl)-1,3-dipropyl-3,7-dihydro-purine-2,6-dione; and
8-Dicyclopropylmethyl-1,3-dipropyl-3,7-dihydro-purine-2,6-dione.

4. The method of claim 1 wherein the A₁ adenosine receptor antagonist is selected from the group consisting of:
3-[4-(2,6-Dioxo-1,3-dipropyl-2,3,6,7-tetrahydro-1H-purin-8-yl)-bicyclo[2.2.2]oct-1-yl]-propionic acid;
7,8-Dihydro-8-isopropyl-2-(4-Hydroxy-bicyclo[2.2.2]oct-1-yl)-4-propyl-1H-imidazo[2,1-i]purin-5-(4H)-one;
7,8-Dihydro-8-ethyl-2-(4-Hydroxy-bicyclo[2.2.2]oct-1-yl)-4-propyl-1H-imidazo[2,1-i]purin-5-(4H)-one;
3-[4-(2,6-Dioxo-1,3-dipropyl-2,3,6,7-tetrahydro-1H-purin-8-yl)-bicyclo[2.2.2]oct-1-yloxy]-propionic acid;
8-(1-Hydroxy-tricyclo[2.2.1.0^{2,6}]hept-3-yl)-1,3-dipropyl-3,7-dihydro-purine-2,6-dione; and
8-(4-Hydroxy-bicyclo[2.2.2]oct-1-yl)-1,3-dipropyl-0,3,7-dihydro-purine-2,6-dione;
8-(3-Oxa-tricyclo[3.2.1.0^{2,4}] oct-6-yl)-1,3-dipropyl-3,7-dihydro-purine-2,6-dione; and
8-Bicyclo[2.2.1]hept-5-en-2-yl-1,3-dipropyl-3,7-dihydro-purine-2,6-dione.

5. The method of claim 1 wherein the A₁ adenosine receptor antagonist is selected from the group consisting of:
3-[4-(2,6-Dioxo-1,3-dipropyl-2,3,6,7-tetrahydro-1H-purin-8-yl)-bicyclo[2.2.2]oct-1-yl]-propionic acid;
7,8-Dihydro-8-isopropyl-2-(4-Hydroxy-bicyclo[2.2.2]oct-1-yl)-4-propyl-1H-imidazo[2,1-i]purin-5-(4H)-one;
7,8-Dihydro-8-ethyl-2-(4-Hydroxy-bicyclo[2.2.2]oct-1-yl)-4-propyl-1H-imidazo[2,1-i]purin-5-(4H)-one;
3-[4-(2,6-Dioxo-1,3-dipropyl-2,3,6,7-tetrahydro-1H-purin-8-yl)-bicyclo[2.2.2]oct-1-yloxy]-propionic acid;
8-(1-Hydroxy-tricyclo[2.2.1.0^{2,6}]hept-3-yl)-1,3-dipropyl-3,7-dihydro-purine-2,6-dione; and
8-(4-Hydroxy-bicyclo[2.2.2]oct-1-yl)-1,3-dipropyl-3,7-dihydro-purine-2,6-dione.

6. The method of claim 1 wherein the Al adenosine receptor antagonist is selected from the group consisting of:
3-(4-(2,6-Dioxo-1,3-dipropyl-2,3,6,7-tetrahydro-1H-purin-8-yl)-bicyclo[2.2.2]oct-1-yl]-propionic acid;
7,8-Dihydro-8-isopropyl-2-(4-Hydroxy-bicyclo[2.2.2]oct-1-yl)-4-propyl-1H-imidazo[2,1-i]purin-5-(4H)-one;
7,8-Dihydro-8-ethyl-2-(4-Hydroxy-bicyclo[2.2.2]oct-1-yl)-4-propyl-1H-imidazo[2,1-i]purin-5-(4H)-one; and
3-[4-(2,6-Dioxo-1,3-dipropyl-2,3,6,7-tetrahydro-1H-purin-8-yl)-bicyclo[2.2.2]oct-1-yloxy]-propionic acid.

7. The method of claim 1 wherein the A₁ adenosine receptor antagonist is 3-[4-(2,6-Dioxo-1,3-dipropyl-2,3,6,7-tetrahydro-1H-purin-8-yl)-bicyclo[2.2.2]oct-1-yl]-propionic acid.

8. The method of claim 1 wherein the Al adenosine receptor antagonist is an antibody.

9. The method of claim 1 or 2 wherein the patient is a human.

10. The method of claim 1 or 2 wherein the A₁ adenosine receptor antagonist is formulated together with a pharmaceutically suitable carrier into a pharmaceutically acceptable composition.

11. The method of claim 10, wherein the patient is a human.

12. The method of claim 10, wherein the patient displays signs or symptoms of a pulmonary disease.

13. The method of claim 10, wherein the pulmonary disease is selected from pulmonary edema, pulmonary hypertension, and a combination thereof.

14. The method of claim 13, wherein the pulmonary edema is accompanied by a condition selected from the group consisting of an imbalance of Starling forces, altered alveolar-capillary membrane permeability, lymphatic insufficiency.

15. The method of claim 13, wherein the pulmonary hypertension is accompanied by a condition selected from the group consisting of pulmonary arterial hypertension, pulmonary hypertension associated with disorders of the respiratory system or hypoxemia, pulmonary venous hypertension, pulmonary hypertension resulting from chronic thrombotic or embolic disease, pulmonary hypertension resulting from disorders directly affecting the pulmonary vasculature.

16. The method of claim 10, wherein the patient displays signs or symptoms of a pulmonary disease characterized by at least one condition selected from the group consisting of global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.

17. The method of claim 1 or 2 wherein the patient is displays signs or symptoms of a pulmonary disease.

18. The method of claim 17, wherein the pulmonary disease is selected from edema and pulmonary hypertension.

19. The method of claim 18, wherein the pulmonary edema is accompanied by a condition selected from the group consisting of an imbalance of Starling forces, altered alveolar-capillary membrane permeability, lymphatic insufficiency.

20. The method of claim 18, wherein the pulmonary hypertension is accompanied by a condition selected from the group consisting of pulmonary arterial hypertension, pulmonary hypertension associated with disorders of the respiratory system or hypoxemia, pulmonary venous hypertension, pulmonary hypertension resulting from chronic thrombotic or embolic disease, pulmonary hypertension resulting from disorders directly affecting the pulmonary vasculature.

21. The method of claim 1 or 2 wherein the patient displays signs or symptoms of a pulmonary disease characterized by at least one condition selected from the group consisting of global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.

22. A method of treating a pulmonary disease, comprising administering to said patient a pharmaceutically effective amount of a pharmaceutical composition comprising an A₁ adenosine antagonist and a pharmaceutically acceptable carrier.

23. The method of claim 22, wherein the pulmonary disease is selected from the group consisting of pulmonary edema, pulmonary hypertension and a combination thereof.

24. The method of claim 23, wherein the pulmonary edema is accompanied by a condition selected from the group consisting of an imbalance of Starling forces, altered alveolar-capillary membrane permeability, lymphatic insufficiency.

25. The method of claim 23, wherein the pulmonary hypertension is accompanied by a condition selected from the group consisting of pulmonary arterial hypertension, pulmonary hypertension associated with disorders of the respiratory system or hypoxemia, pulmonary venous hypertension, pulmonary hypertension resulting from chronic thrombotic or embolic disease, pulmonary hypertension resulting from disorders directly affecting the pulmonary vasculature.

26. The method of claim 22, wherein the patient displays signs or symptoms of a pulmonary disease characterized by at least one condition selected from the group consisting of global pulmonary hypoxia, regional pulmonary hypoxia, pulmonary edema, elevated pulmonary artery pressure, elevated pulmonary vascular resistance, elevated central venous pressure, reduced arterial oxygen saturation, shortness of breath, ‘rales’ and ‘crackles’.
